(19) United States
(12) Patent Application Publication
(10) Pub. No.: US 2004/0087642 A1
(43) Pub. Date: May 6, 2004

(54) Methods of using and compositions comprising a JNK inhibitor for the treatment, prevention, management and/or modification of pain

(76) Inventors: Jerome B. Zeldis, Princeton, NJ (US); Herbert Faleck, West Orange, NJ (US); Donald C. Manning, Bloomsbury, NJ (US)

Correspondence Address: JONES DAY, 222 EAST 41ST STREET, NEW YORK, NY, 10017, US

(21) Appl. No.: 10/693,793

(22) Filed: Oct. 23, 2003

Related U.S. Application Data
(60) Provisional application No. 60/421,104, filed on Oct. 24, 2002.

(51) Int. Cl. A61K 31/416

(52) U.S. Cl. 514/406

(57) ABSTRACT

The present invention relates to methods for treating, preventing, managing and/or modifying pain, comprising administering an effective amount of a JNK Inhibitor to a patient in need thereof. Specific embodiments encompass the administration of a JNK Inhibitor, alone or in combination with a second active agent and/or surgery or physical therapy. Pharmaceutical compositions, single unit dosage forms, and kits suitable for use in methods of the invention are also disclosed.

This application claims the benefit of U.S. provisional application No. 60/421,104, filed Oct. 24, 2002, the contents of which are incorporated by reference herein in their entirety.

### 1. FIELD OF INVENTION

This invention relates to methods for treating, preventing, modifying and/or managing pain and related syndromes, which comprise the administration of a JNK Inhibitor alone or in combination with known therapeutics or therapies. The invention also relates to pharmaceutical compositions comprising a JNK Inhibitor and dosing regimens.

2. Background of the Invention

Pain is the leading symptom of many different disorders and is defined as an unpleasant sensory and emotional experience associated with actual or potential tissue damage or described in terms of such damage. Classification of Chronic Pain, International Association for the Study of Pain (IASP) Task Force on Taxonomy, Merskey H, Bogduk N, eds., IASP Press: Seattle, 209-214, 1994. Becase the perception of pain is highly subjective, it is one of the most difficult pathologies to diagnose and treat effectively. Pain leads to severe impairment of functional ability, which compromises the working, social, and family lives of sufferers. Around five percent of the adult population is estimated to suffer from pain sufficiently severe to cause significant disability. Chojnowska E, Stannard C. Epidemiology of Chronic Pain, Chapter 2, pp 15-26: T. S. Jensen, P. R. Wilson, A. S. C. Rice eds., Clinical Pain Management Chronic Pain, Arnold, London, 2003.

In most pain conditions, there is increased neural input from the periphery. Sensory nerve impulses travel via the axons of primary afferent neurons to the dorsal horn of the spinal cord, where they propagate nerve impulses to dorsal horn neurons by releasing excitatory amino acids and neuropeptides at synapses. Dorsal horn projection neurons process and transfer the information about a peripheral stimuli to the brain via ascending spinal pathways. Mannion, R. J. and Woolf, C. J., Clin. J. of Pain 16:S144-S156 (2000).

The firing of dorsal horn projection neurons is determined not only by the excitatory input they receive, but also by inhibitory input from the spinal cord and higher nerve centers. Several brain regions contribute to descending inhibitory pathways. Nerve fibers from these pathways release inhibitory substances such as endogenous opioids, γ-aminobutyric acid (GABA), and serotonin at synapses with other neurons in the dorsal horn or primary afferent neurons and inhibit nociceptive transmission. Peripheral nerve injury can produce changes in dorsal horn excitability by down-regulating the amount of inhibitory control over dorsal horn neurons through various mechanisms.

Repeated or prolonged stimulation of dorsal horn neurons due to C-nociceptor activation or damaged nerves can cause a prolonged increase in dorsal horn neuron excitability and responsiveness that can last hours longer than the stimulus. Sensitization of the dorsal horn neurons increases their excitability such that they respond to normal input in an exaggerated and extended way. It is now known that such sustained activity in primary afferent C-fibers leads to both morphological and biochemical changes in the dorsal horn which may be difficult to reverse. Several changes in the dorsal horn have been noted to occur with central sensitization: (i) an expansion of the dorsal horn receptive field size so that a spinal neuron will respond to noxious stimuli outside the region normally served by that neuron; (ii) an increase in the magnitude and duration of the response to a given noxious stimulus (hyperalgesia); (iii) a painful response to a normally innocuous stimulus, for example, from a mechanoreceptive primary afferent Aβ-fibre (allodynia); and (iv) the spread of pain to uninjured tissue (referred pain). Koltzenburg, M. Clin. J. of Pain 16:S131-S138 (2000); Mannion, R. J. and Woolf, C. J., Clin. J. of Pain 16:S144-S156 (2000).

Central sensitization may explain, in part, the continuing pain and hyperalgesia that occurs following an injury and may serve an adaptive purpose by encouraging protection of the injury, during the healing phase. Central sensitization however can persist long after the injury has healed thereby supporting chronic pain. Sensitization also plays a key role in chronic pain, helping to explain why it often exceeds the provoking stimulus, both spatially and temporally, and may help explain why established pain is more difficult to suppress than acute pain. Koltzenburg, M. Clin. J. of Pain 16:S131-S138 (2000).

Accordingly, safe and effective methods for the treatment, prevention, modification or management of pain are needed.

2.1 Types of Pain

2.1.1 Nociceptive Pain

Nociceptive pain is elicited when noxious stimuli such as inflammatory chemcial mediators are released following tissue injury, disease, or inflammation and are detected by normally functioning sensory receptors (nociceptors) at the site of injury. Koltzenburg, M. Clin. J. of Pain 16:S131-S138 (2000). Clinical examples of nociceptive pain include, but are not limited to, pain associated with chemical or thermal burns, cuts and contusions of the skin, osteoarthritis, rheumatoid arthritis, tendonitis, and myofascial pain.

Nociceptors (sensory receptors) are distributed throughout the periphery of tissue. They are sensitive to noxious stimuli (e.g., thermal, mechanical, or chemical) which would damage tissue if prolonged. Activation of peripheral nociceptors by such stimuli excites discharges in two distinct types of primary afferent neurons: slowly conducting unmyelinated c-fibers and more rapidly conducting, thinly myelinated Aδ fibers. C-fibers are associated with burning pain and Aδ fibers with stabbing pain. Koltzenburg, M. Clin. J. of Pain 16:S131-S138 (2000); Besson, J. M. Lancet 353:1610-15 (1999); Johnson, B. W. Pain Mechanisms: Anatomy, Physiology and Neurochemistry, Chapter 11 in Practical Management of Pain ed. P. Prithvi Raj. (3^{rd} Ed., Mosby, Inc. St Louis, 2000). Most nociceptive pain involves signaling from both Aδ and c-types of primary afferent nerve fibers.

Peripheral nociceptors are sensitized by inflammatory mediators such as prostaglandin, substance P, bradykinin, histamine, and serotonin, as well as by intense, repeated, or prolonged noxious stimulation. In addition, cytokines and growth factors (e.g., nerve growth factor) can influence neuronal phenotype and function. Besson, J. M. Lancet 353:1610-15 (1999).

When sensitized, nociceptors exhibit a lower activation threshold and an increased rate of firing, which means that they generate nerve impulses more readily and more frequently. Peripheral sensitization of nociceptors plays an important role inspinal cord dorsal horn central sensitization and clinical pain states such as hyperalgesia and allodynia.

Inflammation also appears to have another important effect on peripheral nociceptors. Some C-nociceptors do not normally respond to any level of mechanical or thermal stimuli, and are only activated in the presence of inflammation or in response to tissue injury. Such nociceptors are called “silent” nociceptors, and have been identified in visceral and cutaneous tissue. Besson, J. M. Lancet 353:1610-15 (1999); Koltzenburg, M. Clin. J. of Pain 16:S131-S138 (2000).

Differences in how noxious stimuli are processed across different tissues contribute to the varying characteristics of nociceptive pain. For example, cutaneous pain is often described as a well-localized sharp, prickling, or burning sensation whereas deep somatic pain may be described as diffuse, dull, or an aching sensation. In general, there is a variable association between pain perception and stimulus intensity, as the central nervous system and general experience influence the perception of pain.

2.1.2 Neuropathic Pain

Neuropathic pain reflects injury or impairment of the nervous system, and has been defined by the IASP as “pain initiated or caused by a primary lesion or dysfunction in the nervous system”. Classification of Chronic Pain, International Association for the Study of Pain (IASP) Task Force on Taxonomy, Merskey H, Bogduk N, eds., IASP Press: Seattle, 209-214, 1994. Some neuropathic pain is caused by injury or dysfunction of the peripheral nervous system. As a resultof injury, changes in the expression of key transducer molecules, transmitters, and ion channels occur, leading to altered excitability of peripheral neurons. Johnson, B. W. Pain Mechanisms: Anatomy, Physiology and Neurochemistry, Chapter 11 in Practical Management of Pain ed. P. Prithvi Raj. (3^{rd} Ed., Mosby, Inc., St Louis, 2000). Clinical examples of neuropathic pain include, but are not limited to, pain associated with diabetic neuropathy, postherpetic neuralgia, trigeminal neuralgia, and post-stroke pain.

Neuropathic pain is commonly associated with several distinct characteristics, such as pain which may be continuous or episodic and is described in many ways, such as burning, tingling, prickling, shooting, electric-shock-like, jabbing, squeezing, deep aching, or spasmodic. Paradoxically partial or complete sensory deficit is often present in patients with neuropathic pain who experience diminished perception of thermal and mechanical stimuli. Abnormal or unfamiliar unpleasant sensations (dysaesthesias) may also be present and contribute to suffering. Other features are the ability of otherwise non-noxious stimuli to produce pain (allodynia) or the disproportionate perception of pain in response to supra-threshold stimuli (hyperalgesia). Johnson, B. W. Pain Mechanisms: Anatomy, Physiology and Neurochemistry, Chapter 11 in Practical Management of Pain ed. P. Prithvi Raj. (3^{rd} Ed., Mosby, Inc., St Louis, 2000(; Attal, N. Clin. J ofPain 16:S118-S 130 (2000).

Complex regional pain syndrome (CRPS) is a type of neuropathic pain which usually affects the extremities in the absence (CRPS type I) or presence (CRPS type II) of a nerve injury. CRPS type I encompasses the condition known as reflex sympathetic dystrophy (RSD), CRPS type II encompasses the condition known as causalgia and both types have subsets consistent with sympathetic maintained pain syndrome. In 1993, a special consensus conference of the IASP addressed diagnosis and terminology of the disease, and endorsed the term CRPS with the two subtypes. Subsequent studies and conferences have refined the definitions such that the current guidelines give high sensitivity (0.70) with very high specificity (0.95). Bruehl, et al. Pain 81:147-154 (1999). However, there is still no general agreement on what causes the disease, or how best to treat it. Paice, E., British Medical Journal 310: 1645-1648 (1995).

CRPS is a multi-symptom and multi-system syndrome affecting multiple neural, bone and soft tissues, including one or more extremities, which is characterized by an intense pain. Although it was first described 130 years ago, CRPS remains poorly understood. For example, changes in peripheral and central somatosensory, autonomic, and motor processing, and a pathologic interaction of sympathetic and afferent systems have been proposed as underlying mechanisms. Wasner et al. demonstrated a complete functional loss of cutaneous sympathetic vasoconstrictor activity in an early stage of CRPS with recovery. Wasner G., Heckmann K., Maier C., Arch Neurol 56(5): 613-20 (1999). Kurvers et al. suggested a spinal component to microcirculatory abnormalities at stage I of CRPS, which appeared to manifest itself through a neurogenic inflammatory mechanism. Kurvers H. A., Jacobs M. J., Beuk R. J., Pain 60(3): 333-40 (1995). The cause of vascular abnormalities is unknown, and debate still surrounds the question of whether the sympathetic nervous system (SNS) is involved in the generation of these changes.

The actual incidence of CRPS in the U.S. is unknown, and limited information is available about the epidemiology of the disease. Both sexes are affected, but the incidence of the syndrome is higher in women. The syndrome may occur in any age group, including the pediatric population. Schwartzman R. J., Curr Opin Neurol Neurosurg 6(4): 531-6 (1993). Various causes that have led to CRPS include, but are not limited to, head injury, stroke, polio, tumor, trauma, amylotrophic lateral sclerosis (ALS), myocardial infarction, polymyalgia rheumatica, operative procedure, brachial plexopathy, cast/splint immobilization, minor extremity injury and malignancy.

Symptoms of CRPS include, but are not limited to, pain, autonomic dysfunction, edema, movement disorder, dystrophy, and atrophy. Schwartzman R. J., N Engl J Med 343(9): 654-6 (2000). The pain is described as extremely severe and unrelenting, often with a burning character. Ninety percent of all CRPS patients complain of spontaneous burning pain and allodynia, which refers to pain with light touch. Much of the difficulty clinicians have with this syndrome is the fact that pain may be far worse than what would be expected based on physical findings. Id. Pain is also accompanied by swelling and joint tenderness, increased sweating, sensitivity to temperature and light touch, as well as color change to the skin. In fact, the diagnosis of CRPS cannot be made on reports of pain alone. Patients must have signs and symptoms of sensory abnormalities as well as vascular dysfunction accompanied by excessive sweating, edema or trophic changes to the skin.

As metnioned above, the IASP has divided CRPS into two types, namely CRPS type I (also referred to as RSD) and CRPS type II (also referred to as causalgia). These two types are differentiated mainly based upon whether the inciting incident included a definable nerve injury. CRPS type I occurs after an initial noxious event other than a nerve injury. CRPS type II occurs after nerve injury. CRPS is further divided into distinct stages in its development and manifestation. However, the course of the disease seems to be so unpredictable between various patients that staging is not always clear or helpful in treatment. Schwartzman R. J., N Engl J Med 343(9): 654 (2000).

In stage I, or “early RSD,” pain is more severe than would be expected from the injury, and it has a burning or aching quality. It may be increased by dependency of the limb, physical contact, or emotional upset. The affected area typically becomes edematous, may be hyperthermic or hypothermic, and may show increased nail and hair growth. Radiographs may show early bony changes. Id.

In stage II, or “established RSD,” edematous tissue becomes indurated. Skin typically becomes cool and hyperhidrotic with livedo reticularis or cyanosis. Hair may be lost, and nails become ridged, cracked, and brittle. Hand dryness becomes prominent, and atrophy of skin and subcutaneous tissues becomes noticeable. Pain remains the dominant feature. It is usually constant and is increased by any stimulus to the affected area. Stiffniess develops at this stage. Radiographs may show diffuse osteoporosis. Id.

In stage III, or “late RSD,” pain spreads proximally. Although it may diminish in intensity, pain remains a prominent feature. Flare-ups may occur spontaneously. Irreversible tissue damage occurs, and the skin is typically thin and shiny. Edema is absent, but contractures may occur. X-ray films typically indicate marked bone demineralization. Id.

In all stages of CRPS, patients endure severe chronic pain and most patients are sleep deprived. CRPS has significant morbidity and thus raising awareness of the disease is important. Early and effective treatment may lessen the effect of CRPS in some individuals. William D. Dzwierzynski et al., Hand Clinics Vol 10 (1): 29-44 (1994).

2.1.3 Other Types of Pain

Visceral pain has been conventionally viewed as a variant of somatic pain, but may differ in neurological mechanisms. Visceral pain is also thought to involve silent nociceptors, visceral afferent fibers that only become activated in the presence of inflammation. Cervero, F. and Laird J. M. A., Lancet 353:2145-48 (1999).

Certain clinical characteristics are peculiar to visceral pain: (i) it is not evoked from all viscera and not always linked to visceral injury; (ii) it is often diffuse and poorly localized, due to the organization of visceral nociceptive pathways in the central nervous system (CNS), particularly the absence of a separate visceral sensory pathway and the low proportion of visceral afferent nerve fibers; (iii) it is sometimes referred to other non-visceral structures; and (iv) it is associated with motor and autonomic reflexes, such as nausea. Johnson, B. W. Pain Mechanisms: Anatomy, Physiology and Neurochemistry, Chapter 11 in Practical Management of Pain ed. P. Prithvi Raj. (3 Ed., Mosby, Inc., St Louis, 2000); Cervero, F. and Laird J. M. A., Lancet 353:2145-48 (1999).

Headaches can be classified as primary and secondary headache disorders. The pathophysiology of the two most common primary disorders, migraine and tension-type headache, is complex and not fully understood. Recent studies indicate that nociceptive input to the CNS may be increased due to the activation and sensitization of peripheral nociceptors, and the barrage of nociceptive impulses results in the activation and sensitization of second- and third-order neurons in the CNS. Thus, it is likely that central sensitization plays a role in the initiation and maintenance of migraine and tension-type headache. Johnson, B. W. Pain Mechanisms: Anatomy, Physiology and Neurochemistry, Chapter 11 in Practical Management of Pain ed. P. Prithvi Raj. (3^{rd} Ed., Mosby, Inc., St Louis, 2000).

Post-operative pain, such as that resulting from trauma to tissue caused during surgery, produces a barrage of nociceptive input. Following surgery, there is an inflammatory response at the site of injury involving cytokines, neuropeptides and other inflammatory mediators. These chemical are responsible for the sensitization and increased responsiveness to external stimuli, resulting in, for example, lowering of the threshold and an increased response to supra-threshold stimuli. Together, these processes result in peripheral and central sensitization. Johnson, B. W. Pain Mechanisms: Anatomy, Physiology and Neurochemistry, Chapter 11 in Practical Management of Pain ed. P. Prithvi (Raj. 3^{rd} Ed., Mosby, Inc., St Louis, 2000).

Mixed pain is chronic pain that has nociceptive and neuropathic components. For example, a particular pain can be initiated through one pain pathway and sustained through a different pain pathway. Examples of mixed pain states include, but are not limited to, cancer pain and low back pain.

2.2 Current Treatments for Pain

Current treatment for CRPS related pain in particular and chronic pain in general includes pain management and extensive physical therapy, which can help to prevent edema and joint contractures and can also help to minimize pain. Often, medication and neural blockade are used to help with the severe pain. Regional neural blockade is performed using Bier blocks with a variety of agents, including local anesthetics, bretylium, steroids, calcitonin, reserpine, and guanethidine. Perez, R. S., et al., J. Pain Symptom Manage 21(6): 511-26 (2001). Specific, selective sympathetic ganglia neural blockade is performed for both diagnostic and therapeutic purposes. The rationale for selective neural blockade is to interrupt the sympathetic nervous system and reduce the activation of the sensory nerves. Patients who fail well-controlled neural blockade treatment may have pain that is sympathetic-independent Once refractory to neural blockade, pain is typically lifelong and may be severe enough to be debilitating. Id.

Medications presently used during the treatment of chronic pain in general include calcium channel blockers, muscle relaxants, non-narcotic analgesics, opioid analgesics, and systemic corticosteroids. However, patients rarely obtain complete pain relief. Moreover, because the mechanisms of pain and autonomic dysfunction are poorly understood, the treatments are completely empirical. Therefore, there remains a need for safe and effective methods of treating and managing pain.

### 3. SUMMARY OF THE INVENTION

The present invention relates to methods for treating or preventing pain, comprising administering to a patient in need thereof a therapeutically or prophylactically effective amount of a JNK Inhibitor. The invention also relates to methods for managing (e.g., lengthening the time of remission) pain, which comprise administering to a patient in need of such management a therapeutically or prophylactically effective amount of a JNK Inhibitor. The invention further relates to methods for modifying pain, which comprise administering to a patient in need thereof a therapeutically or prophylactically effective amount of a JNK Inhibitor.

Another embodiment of the invention encompasses the use of one or more JNK Inhibitors with another therapeutic useful for the treatment, prevention, management and/or modification of pain such as, but not limited to, an antidepressant, antihypertensive, anxiolytic, calcium channel blocker, muscle relaxant, non-narcotic analgesic, anti-inflammatory agent, cox-2 inhibitor, alpha-adrenergic receptor agonist or antagonist, ketamine, anesthetics, immunomodulatory agent, immunosuppressive agent, corticosteroid, hyperbaric oxygen, anticonvulsant, an IMiD®, a SelCID®, or a combination thereof.

Yet another embodiment of the invention encompasses the use of one or more JNK Inhibitors in combination with conventional therapies used to treat, prevent, manage and/or modify pain including, but not limited to, surgery, interventional procedures (e.g., neural blockade), physical therapy, and psychological therapy.

The invention further encompasses pharmaceutical compositions, single unit dosage forms, and kits suitable for use in treating, preventing, managing and/or modifying pain, which comprise a therpeutically or prophylactically effective amount of a JNK Inhibitor.

3.1 Definitions

As used herein, the term “patient” means an animal (e.g., cow, horse, sheep, pig, chicken, turkey, quail, cat, dog, mouse, rat, rabbit or guinea pig), preferably a mammal such as a non-primate and a primate (e.g., monkey and human), most preferably a human.

“Alkyl” means a saturated straight chain or branched non-cyclic hydrocarbon having from 1 to 10 carbon atoms. “Lower alkyl” means alkyl, as defined above, having from 1 to 4 carbon atoms. Representative saturated straight chain alkyls include -methyl, -ethyl, -n-propyl, -n-butyl, -n-pentyl, -n-hexyl, -n-heptyl, -n-octyl, -n-nonyl and -n-decyl; while saturated branched alkyls include -isopropyl, -sec-butyl, -isobutyl, -tert-butyl, -isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimtheylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl and the like.

An “alkenyl group” or “alkylidene” mean a straight chain or branched non-cyclic hydrocarbon having from 2 to 10 carbon atoms and including at least one carbon-carbon double bond. Representative straight chain and branched (C₂-C₁₀)alkenyls include -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, -1-hexenyl, -2-hexenyl, -3-hexenyl, -1-heptenyl, -2-heptenyl, -3-heptenyl, -1-octenyl, -2-octenyl, -3-octenyl, -1-nonenyl, -2-nonenyl, -3-nonenyl, -1-decenyl, -2-decenyl, -3 decenyl and the like. An alkenyl group can be unsubstituted or substituted. A “cyclic alkylidene” is a ring having from 3 to 8 carbon atoms and including at least one carbon-carbon double bond, wherein the ring can have from 1 to 3 heteroatoms.

An “alkynyl group” means a straight chain or branched non-cyclic hydrocarbon having from 2 to 10 carbon atoms and including at lease one carbon-carbon triple bond. Representative straight chain and branched —(C₂-C₁₀)alkynyls include -acetylenyl, -propynyl, -1-butynyl, -2-butynyl, -1-pentynyl, -2-pentynyl, -3-methyl-1-butynyl, -4-pentynyl, -1-hexynyl, -2-hexynyl, -5-hexynyl, -1-heptynyl, -2-heptynyl, -6-heptynyl, -1-octynyl, -2-octynyl, -7-octynyl, -1-nonynyl, -2-nonynyl, -8-nonynyl, -1-decynyl, -2-decynyl, -9-decynyl, and the like. An alkynyl group can be unsubstituted or substituted.

The terms “Halogen” and “Halo” mean fluorine, chlorine, bromine or iodine.

“Haloalkyl” means an alkyl group, wherein alkyl is defined above, substituted with one or more halogen atoms.

“Keto” means a carbonyl group (i.e., C═O).

“Acyl” means an —C(O)alkyl group, wherein alkyl is defined above, including —C(O)CH₃, —C(O)CH₂CH₃, —C(O)(CH₂)₂CH₃, —C(O)(CH₂)₃CH₃, —C(O)(CH₂)₄CH₃, —C(O)(CH₂)₅CH₃, and the like.

“Acyloxy” means an —OC(O)alkyl group, wherein alkyl is defined above, including —OC(O)CH₃, —OC(O)CH₂CH₃, —OC(O)(CH₂)₂CH₃, —OC(O)(CH₂)₃CH₃, —OC(O)(CH₂)₄CH₃, —OC(O)(CH₂)₅CH₃, and the like.

“Ester” means and —C(O)Oalkyl group, wherein alkyl is defined above, including —C(O)OCH₃, —C(O)OCH₂CH₃, —C(O)O(CH₂)₂CH₃, —C(O)O(CH₂)₃CH₃, —C(O)O(CH₂)₄CH₃, —C(O)O(CH₂)₅CH₃, and the like.

“Alkoxy” means —O-(alkyl), wherein alkyl is defined above, including —OCH₃, —OCH₂CH₃, —O(CH₂)₂CH₃, —O(CH₂)₃CH₃, —O(CH₂)₄CH₃, —O(CH₂)₅CH₃, and the like. “Lower alkoxy” means —O-(lower alkyl), wherein lower alkyl is as described above.

“Alkoxyalkoxy” means —O-(alkyl)-O-(alkyl), wherein each alkyl is independently an alkyl group defined above, including —OCH₂OCH₃, —OCH₂CH₂OCH₃, —OCH₂CH₂OCH₂CH₃, and the like.

“Alkoxycarbonyl” means —C(═O)O-(alkyl), wherein alkyl is defined above, including —C(═O)O—CH₃, —C(═O)O—CH₂CH₃, —C(═O)O—(CH₂)₂CH₃, —C(═O)O—(CH₂)₃CH₃, —C(═O)O—(CH₂)₄CH₃, —C(═O)O—(CH₂)₅CH₃, and the like.

“Alkoxycarbonylalkyl” means -(alkyl)-C(═O)O-(alkyl), wherein each alkyl is independently defined above, including —CH₂—C(═O)O—CH₃, —CH₂—C(═O)O—CH₂CH₃, —CH₂—C(═O)O—(CH₂)₂CH₃, —CH₂—C(═O)O—(CH₂)₃CH₃, —CH₂—C(═O)O—(CH₂)₄CH₃, —CH₂—C(═O)O—(CH₂)₅CH₃, and the like.

“Alkoxyalkyl” means -(alkyl)-O-(alkyl), wherein each alkyl is independently an alkyl group defined above, including —CH₂OCH₃, —CH₂OCH₂CH₃, —(CH₂)₂OCH₂CH₃, —(CH₂)₂O(CH₂)₂CH₃, and the like.

“Aryl” means a carbocyclic aromatic group containing from 5 to 10 ring atoms. Representative examples include, but are not limited to, phenyl, tolyl, anthracenyl, fluorenyl, indenyl, azulenyl, pyridinyl and naphthyl, as well as benzo-fused carbocyclic moieties including 5,6,7,8-tetrahydronaphthyl. A carbocyclic aromatic group can be unsubstituted or substituted. In one embodiment, the carbocyclic aromatic group is a phenyl group.

“Aryloxy” means —O-aryl group, wherein aryl is as defined above. An aryloxy group can be unsubstituted or substituted. In one embodiment, the aryl ring of an aryloxy group is a phenyl group

“Arylalkyl” means -(alkyl)-(aryl), wherein alkyl and aryl are as defined above, including —(CH₂)phenyl, —(CH₂)₂phenyl, —(CH₂)₃phenyl, —CH(phenyl)₂, —CH(phenyl)₃, —(CH₂)tolyl, —(CH₂)anthracenyl, —(CH₂)fluorenyl, —(CH₂)indenyl, —(CH₂)azulenyl, —(CH₂)pyridinyl, —(CH₂)naphthyl, and the like.

“Arylalkyloxy” means —O-(alkyl)-(aryl), wherein alkyl and aryl are defined above, including —O—(CH₂)₂phenyl, —O—(CH₂)₃phenyl, —O—CH(phenyl)₂, —O—CH(phenyl)₃, —O—(CH₂)tolyl, —O—(CH₂)anthracenyl, —O—(CH₂)fluorenyl, —O—(CH₂)indenyl, —O—(CH₂)azulenyl, —O—(CH₂)pyridinyl, —O—(CH₂)naphthyl, and the like.

“Aryloxyalkyl” means -(alkyl)-O-(aryl), wherein alkyl and aryl are defined above, including —CH₂—O-(phenyl), —(CH₂)₂—O-phenyl, —(CH₂)₃—O-phenyl, —(CH₂)—O-tolyl, —(CH₂)—O-anthracenyl, —(CH₂)—O-fluorenyl, —(CH₂)—O-indenyl, —(CH₂)—O-azulenyl, —(CH₂)—O-pyridinyl, —(CH₂)—O-naphthyl, and the like.

“Cycloalkyl” means a monocyclic or polycyclic saturated ring having carbon and hydrogen atoms and having no carbon-carbon multiple bonds. Examples of cycloalkyl groups include, but are not limited to, (C₃-C₇)cycloalkyl groups, including cyclopropyl, cyclobutyl, cyclopentyl, cyclohexyl, and cycloheptyl, and saturated cyclic and bicyclic terpenes. A cycloalkyl group can be unsubstituted or substituted. In one embodiment, the cycloalkyl group is a monocyclic ring or bicyclic ring.

“Cycloalkyloxy” means —O-(cycloalkyl), wherein cycloalkyl is defined above, including —O-cyclopropyl, —O-cyclobutyl, —O-cyclopentyl, —O-cyclohexyl, —O-cycloheptyl and the like.

“Cycloalkylalkyloxy” means —O-(alkyl)-(cycloalkyl), wherein cycloalkyl and alkyl are defined above, including —O—CH₂-cyclopropyl, —O—(CH₂)₂-cyclopropyl, —O—(CH₂)₃-cyclopropyl, —O—(CH₂)₄-cyclopropyl, O—CH₂-cyclobutyl, O—CH₂-cyclopentyl, —O—CH₂-cyclohexyl, O—CH₂-cycloheptyl, and the like.

“Aminoalkoxy” means —O-(alkyl)-NH₂, wherein alkyl is defined above, such as —O—CH₂—NH₂, —O—(CH₂)₂—NH₂, —O—(CH₂)₃—NH₂, —O—(CH₂)₄—NH₂, —O—(CH₂)₅—NH₂, and the like.

“Mono-alkylamino” means —NH(alkyl), wherein alkyl is defined above, such as —NHCH₃, —NHCH₂CH₃, —NH(CH₂)₂CH₃, —NH(CH₂)₃CH₃, —NH(CH₂)₄CH₃, —NH(CH₂)₅CH₃, and the like.

“Di-alkylamino” means —N(alkyl)(alkyl), wherein each alkyl is independently an alkyl group defined above, including —N(CH₃)₂, —N(CH₂CH₃)₂, —N((CH₂)₂CH₃)₂, —N(CH₃)(CH₂CH₃), and the like.

“Mono-alkylaminoalkoxy” means —O-(alkyl)-NH(alkyl), wherein each alkyl is independently an alkyl group defined above, including —O—(CH₂)—NHCH₃, —O—(CH₂)—NHCH₂CH₃, —O—(CH₂)—NH(CH₂)₂CH₃, —O—(CH₂)—NH(CH₂)₃CH₃, —O—(CH₂)—NH(CH₂)₄CH₃, —O—(CH₂)—NH(CH₂)₅CH₃, —O—(CH₂)₂—NHCH₃, and the like.

“Di-alkylaminoalkoxy” means —O-(alkyl)-N(alkyl)(alkyl), wherein each alkyl is independently an alkyl group defined above, including —O—(CH₂)—N(CH₃)₂, —O—(CH₂)—N(CH₂CH₃)₂, —O—(CH₂)—N((CH₂)₂CH₃)₂, —O—(CH₂)—N(CH₃)(CH₂CH₃), and the like.

“Arylamino”means —NH(aryl), wherein aryl is defined above, including —NH(phenyl), —NH(tolyl), —NH(anthracenyl), —NH(fluorenyl), —NH(indenyl), —NH(azulenyl), —NH(pyridinyl), —NH(naphthyl), and the like.

“Arylalkylamino” means —NH-(alkyl)-(aryl), wherein alkyl and aryl are defined above, including —NH—CH₂— (phenyl), —NH—CH₂— (tolyl), —NH—CH₂— (anthracenyl), —NH—CH₂— (fluorenyl), —NH—CH₂— (indenyl), —NH—CH₂— (azulenyl), —NH—CH₂— (pyridinyl), —NH—CH₂— (naphthyl), —NH—(CH₂)₂-(phenyl) and the like.

“Alkylamino” means mono-alkylamino or di-alkylamino as defined above, such as —N(alkyl)(alkyl), wherein each alkyl is independently an alkyl group defined above, including —N(CH₃)₂, —N(CH₂CH₃)₂, —N((CH₂)₂CH₃)₂, —N(CH₃)(CH₂CH₃) and —N(alkyl)(alkyl), wherein each alkyl is independently an alkyl group defined above, including —N(CH₃)₂, —N(CH₂CH₃)₂, —N((CH₂)₂CH₃)₂, —N(CH₃)(CH₂CH₃) and the like.

“Cycloalkylamino” means —NH-(cycloalkyl), wherein cycloalkyl is as defined above, including —NH-cyclopropyl, —NH-cyclobutyl, —NH-cyclopentyl, —NH-cyclohexyl, —NH-cycloheptyl, and the like.

“Carboxyl” and “carboxy” mean —COOH.

“Cycloalkylalkylamino” means —NH-(alkyl)-(cycloalkyl), wherein alkyl and cycloalkyl are defined above, including —NH—CH₂-cyclopropyl, —NH—CH₂-cyclobutyl, —NH—CH₂-cyclopentyl, —NH—CH₂-cyclohexyl, —NH—CH₂-cycloheptyl, —NH—(CH₂)₂-cyclopropyl and the like.

“Aminoalkyl” means -(alkyl)-NH₂, wherein alkyl is defined above, including CH₂—NH₂, —(CH₂)₂—NH₂, —(CH₂)₃—NH₂, —(CH₂)₄—N1₂, —(CH₂)₅—NH₂ and the like.

“Mono-alkylaminoalkyl” means -(alkyl)-NH(alkyl), wherein each alkyl is independently an alkyl group defined above, including —CH₂—NH—CH₃, —CH₂—NHCH₂CH₃, —CH₂—NH(CH₂)₂CH₃, —CH₂—NH(CH₂)₃CH₃, —CH₂—NH(CH₂)₄CH₃, —CH₂—NH(CH₂)₅CH₃, —(CH₂)₂—NH—CH₃, and the like.

“Di-alkylaminoalkyl” means -(alkyl)-N(alkyl)(alkyl), wherein each alkyl is independently an alkyl group defined above, including —CH₂—N(CH₃)₂, —CH₂—N(CH₂CH₃)₂, —CH₂—N((CH₂)₂CH₃)₂, —CH₂—N(CH₃)(CH₂CH₃), —(CH₂)₂—N(CH₃)₂, and the like.

“Heteroaryl” means an aromatic heterocycle ring of 5- to 10 members and having at least one heteroatom selected from nitrogen, oxygen and sulfur, and containing at least 1 carbon atom, including both mono- and bicyclic ring systems. Representative heteroaryls are triazolyl, tetrazolyl, oxadiazolyl, pyridyl, furyl, benzofuranyl, thiophenyl, benzothiophenyl, quinolinyl, pyrrolyl, indolyl, oxazolyl, benzoxazolyl, imidazolyl, benzimidazolyl, thiazolyl, benzothiazolyl, isoxazolyl, pyrazolyl, isothiazolyl, pyridazinyl, pyrimidinyl, pyrazinyl, triazinyl, cinnolinyl, phthalazinyl, quinazolinyl, pyrimidyl, oxetanyl, azepinyl, piperazinyl, morpholinyl, dioxanyl, thietanyl and oxazolyl.

“Heteroarylalkyl” means -(alkyl)-(heteroaryl), wherein alkyl and heteroaryl are defined above, including —CH₂-triazolyl, —CH₂-tetrazolyl, —CH₂-oxadiazolyl, —CH₂-pyridyl, —CH₂-furyl, —CH₂-benzofuranyl, —CH₂-thiophenyl, —CH₂-benzothiophenyl, —CH₂-quinolinyl, —CH₂-pyrrolyl, —CH₂-indolyl, —CH₂-oxazolyl, —CH₂-benzoxazolyl, —CH₂-imidazolyl, —CH₂-benzimidazolyl, —CH₂-thiazolyl, —CH₂-benzothiazolyl, —CH₂-isoxazolyl, —CH₂-pyrazolyl, —CH₂-isothiazolyl, —CH₂-pyridazinyl, —CH₂-pyrimidinyl, —CH₂-pyrazinyl, —CH₂-triazinyl, —CH₂-cinnolinyl, —CH₂-phthalazinyl, —CH₂-quinazolinyl, —CH₂-pyrimidyl, —CH₂-oxetanyl, —CH₂-azepinyl, —CH₂-piperazinyl, —CH₂-morpholinyl, —CH₂-dioxanyl, —CH₂-thietanyl, —CH₂-oxazolyl, —(CH₂)₂-triazolyl, and the like.

“Heterocycle” means a 5- to 7-membered monocyclic, or 7- to 10-membered bicyclic, heterocyclic ring which is either saturated, unsaturated, and which contains from 1 to 4 heteroatoms independently selected from nitrogen, oxygen and sulfur, and wherein the nitrogen and sulfur heteroatoms can be optionally oxidized, and the nitrogen heteroatom can be optionally quaternized, including bicyclic rings in which any of the above heterocycles are fused to a benzene ring. The heterocycle can be attached via any heteroatom or carbon atom. Heterocycles include heteroaryls as defined above. Representative heterocycles include morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

“Heterocycle fused to phenyl” means a heterocycle, wherein heterocycle is defined as above, that is attached to a phenyl ring at two adjacent carbon atoms of the phenyl ring.

“Heterocycloalkyl” means -(alkyl)-(heterocycle), wherein alkyl and heterocycle are defined above, including —CH₂-morpholinyl, —CH₂-pyrrolidinonyl, —CH₂-pyrrolidinyl, —CH₂-piperidinyl, —CH₂-hydantoinyl, —CH₂-valerolactamyl, —CH₂-oxiranyl, —CH₂-oxetanyl, —CH₂-tetrahydrofuranyl, —CH₂-tetrahydropyranyl, —CH₂-tetrahydropyridinyl, —CH₂-tetrahydroprimidinyl, —CH₂-tetrahydrothiophenyl, —CH₂-tetrahydrothiopyranyl, —CH₂-tetrahydropyrimidinyl, —CH₂-tetrahydrothiophenyl, —CH₂-tetrahydrothiopyranyl, and the like.

The term “substituted” as used herein means any of the above groups (i.e., aryl, arylalkyl, heterocycle and heterocycloalkyl) wherein at least one hydrogen atom of the moiety being substituted is replaced with a substituent. In one embodiment, each carbon atom of the group being substituted is substituted with no more that two substituents. In another embodiment, each carbon atom of the group being substituted is substituted with no more than one substituent. In the case of a keto substituent, two hydrogen atoms are replaced with an oxygen which is attached to the carbon via a double bond. Substituents include halogen, hydroxyl, alkyl, haloalkyl, mono- or di-substituted aminoalkyl, alkyloxyalkyl, aryl, arylalkyl, heterocycle, heterocycloalkyl, —NRₐR_{b}, —NRₐC(═O)R_{b}, —NRₐC(═O)NRₐR_{b}, —NRₐC(═O)OR_{b}, —NRₐSO₂R_{b}, —ORₐ, —C(═O)RₐC(═O)ORₐ—C(═O)NRₐR_{b}, —OC(═O)Rₐ, —OC(═O)ORₐ, —OC(═O)NRₐR_{b}, —NRₐSO₂R_{b}, or a radical of the formula —Y—Z—Rₐ where Y is alkanediyl, or a direct bond, Z is —O—, —S—, —N(R_{b})—, —C(═O)—, —C(═O)O—, —OC(═O)—, —N(R_{b})C(═O)—, —C(═O)N(R_{b})— or a direct bond, wherein Rₐ and R_{b}are the same or different and independently hydrogen, amino, alkyl, haloalkyl, aryl, arylalkyl, heterocycle, or heterocylealkyl, or wherein Rₐ and R_{b}taken together with the nitrogen atom to which they are attached form a heterocycle.

“Haloalkyl” means alkyl, wherein alkyl is defined as above, having one or more hydrogen atoms replaced with halogen, wherein halogen is as defined above, including —CF₃, —CHF₂, —CH₂F, —CBr₃, —CHBr₂, —CH₂Br, —CCl₃, —CHCl₂, —CH₂Cl, —CI₃, —CHI₂, —CH₂I, —CH₂—CF₃, —CH₂—CHF₂, —CH₂—CH₂F, —CH₂—CBr₃, —CH₂—CHBr₂, —CH₂—CH₂Br, —CH₂—CCl₃, —CH₂—CHCl₂, —CH₂—CH₂Cl, —CH₂—CI₃, —CH₂—CHI₂, —CH₂—CH₂I, and the like.

“Hydroxyalkyl” means alkyl, wherein alkyl is as defined above, having one or more hydrogen atoms replaced with hydroxy, including —CH₂OH, —CH₂CH₂OH, —(CH₂)₂CH₂OH, —(CH₂)₃CH₂OH, —(CH₂)₄CH₂OH, —(CH₂)₅CH₂OH, —CH(OH)—CH₃, CH₂CH(OH)CH₃, and the like.

“Hydroxy” means —OH.

“Sulfonyl” means —SO₃H.

“Sulfonylalkyl” means —SO₂— (alkyl), wherein alkyl is defined above, including —SO₂—CH₃, —SO₂—CH₂CH₃, —SO₂—(CH₂)₂CH₃, —SO₂—(CH₂)₃CH₃, —SO₂—(CH₂)₄CH₃, —SO₂—(CH₂)₅CH₃, and the like.

“Sulfinylalkyl” means —SO-(alkyl), wherein alkyl is defined above, including —SO—CH₃, —SO—CH₂CH₃, —SO—(CH₂)₂CH₃, —SO—(CH₂)₃CH₃, —SO—(CH₂)₄CH₃, —SO—(CH₂)₅CH₃, and the like.

“Sulfonamidoalkyl” means —NHSO₂— (alkyl), wherein aklyl is defined above, including —NHSO₂—CH₃, —NHSO₂—CH₂CH₃, —NHSO₂—(CH₂)₂CH₃, —NHSO₂—(CH₂)₃CH₃, —NHSO₂—(CH₂)₄CH₃, —NHSO₂—(CH₂)₅CH₃, and the like.

“Thioalkyl” means —S-(alkyl), wherein alkyl is defined above, including —S—CH₃, —S—CH₂CH₃, —S—(CH₂)₂CH₃, —S—(CH₂)₃CH₃, —S—(CH₂)₄CH₃, —S—(CH₂)₅CH₃, and the like.

As used herein, the term “JNK Inhibitor” encompasses, but is not limited to, compounds disclosed herein. Without being limited by theory, specific JNK Inhibitors capable of inhibiting the activity of JNK in vitro or in vivo. The JNK Inhibitor can be in the form of a pharmaceutically acceptable salt, free base, solvate, hydrate, stereoisomer, clathrate or prodrug thereof. Such inhibitory activity can be determined by an assay or animal model well-known in the art including those set forth in Section 5. In one embodiment, the JNK Inhibitor is a compound of structure (I)-(III).

“JNK” means a protein or an isoform thereof expressed by a JNK 1, JNK 2, or JNK 3 gene (Gupta, S., Barrett, T., Whitmarsh, A. J., Cavanagh, J., Sluss, H. K., Derijard, B. and Davis, R. J. The EMBO J. 15:2760-2770 (1996)).

As used herein, the phrase “an effective amount” when used in connection with a JNK Inhibitor means an amount of the JNK Inhibitor that is useful for for treating, preventing, managing and/or modifying pain.

As used herein, the phrase “an effective amount” when used in connection with another therpeutic or prophylactic agent means an amount of the other therapeutic or prophylactic agent that is useful for for treating, preventing, managing and/or modifying pain when administered while the JNK Inhibitor exerts its therapeutic or prophylactic activity.

As used herein, the term “pharmaceutically acceptable salt(s)” refers to a salt prepared from a pharmaceutically acceptable non-toxic acid or base including an inorganic acid and base and an organic acid and base. Suitable pharmaceutically acceptable base addition salts of the JNK Inhibitor include, but are not limited to metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, phosphoric, sulfuric, and methanesulfonic acids. Examples of specific salts thus include hydrochloride and mesylate salts. Others are well-known in the art, see for example, Remington's Pharmaceutical Sciences, 18^{th} eds., Mack Publishing, Easton Pa. (1990) or Remington: The Science and Practice of Pharmacy, 19^{th} eds., Mack Publishing, Easton Pa. (1995).

As used herein and unless otherwise indicated, the term “polymorph” means a particular crystalline arrangement of the JNK Inhibitor. Polymorphs can be obtained through the use of different work-up conditions and/or solvents. In particular, polymorphs can be prepared by recrystallization of a JNK Inhibitor in a particular solvent.

As used herein and unless otherwise indicated, the term “prodrug” means a JNK Inhibitor derivative that can hydrolyze, oxidize, or otherwise react under biological conditions (in vitro or in vivo) to provide an active compound, particularly a JNK Inhibitor. Examples of prodrugs include, but are not limited to, derivatives and metabolites of a JNK Inhibitor that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Preferably, prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger 's Medicinal Chemistry and Drug Discovery 6^{th} ed. (Donald J. Abraham ed., 2001, Wiley) and Design and Application of Prodrugs (H. Bundgaard ed., 1985, Harwood Academic Publishers Gmfh).

As used herein and unless otherwise indicated, the term “optically pure” or “stereomerically pure” means one stereoisomer of a compound is substantially free of other stereoisomers of that compound. For example, a stereomerically pure compound having one chiral center will be substantially free of the opposite enantiomer of the compound. A stereomerically pure a compound having two chiral centers will be substantially free of other diastereomers of the compound. A typical stereomerically pure compound comprises greater than about 80% by weight of one stereoisomer of the compound and less than about 20% by weight of other stereoisomers of the compound, more preferably greater than about 90% by weight of one stereoisomer of the compound and less than about 10% by weight of the other stereoisomers of the compound, even more preferably greater than about 95% by weight of one stereoisomer of the compound and less than about 5% by weight of the other stereoisomers of the compound, and most preferably greater than about 97% by weight of one stereoisomer of the compound and less than about 3% by weight of the other stereoisomers of the compound.

As used herein, the terms “complex regional pain syndrome,” “CRPS” and “CRPS and related syndromes” mean a chronic pain disorder characterized by one or more of the following: pain, whether spontaneous or evoked, including allodynia (painful response to a stimulus that is not usually painful) and hyperalgesia (exaggerated response to a stimulus that is usually only mildly painful); pain that is disproportionate to the inciting event (e.g., years of severe pain after an ankle sprain); regional pain that is not limited to a single peripheral nerve distribution; and autonomic dysregulation (e.g., edema, alteration in blood flow and hyperhidrosis) associated with trophic skin changes (hair and nail growth abnormalities and cutaneous ulceration). Unless otherwise indicated, the terms “complex regional pain syndrome” and “CRPS” include: type I, encompassing the condition known as reflex sympathetic dystrophy (RSD), which occurs after an initial noxious event other than a nerve injury; type II, encompassing the condition known as causalgia, which occurs after nerve injury; acute stage (usually hyperthermic phase of 2-3 months); dystrophic phase (showing vasomotor instability for several months); atrophic phase (usually cold extremity with atrophic changes); reflex neurovascular dystrophy; reflex dystrophy; sympathetic maintained pain syndrome; Sudeck atrophy of bone; algoneurodystrophy; shoulder hand syndrome; post-traumatic dystrophy; trigeminal neuralgia; post herpetic neuralgia; cancer related pain; phantom limb pain; fibromyalgia; chronic fatigue syndrome; radiculopathy; and other painful neuropathic conditions, e.g., diabetic neuropathy, luetic neuropathy, painful neuropathy induced iatrogenically by drugs such as vincristine, velcade or thalidomide.

As used herein, unless otherwise specified, the term “treating pain” refers to the administration of a JNK Inhibitor, optionally in combination with another active agent or other therapy, after the onset of a symptom of pain, whereas “preventing pain”refers to the administration of a JNK Inhibitor, optionally in combination with another active agent or other therapy, prior to the onset of a symptom of pain, particularly to patients at risk of experiencing pain. Examples of patients at risk of experiencing pain include, but are not limited to, those who have incidents of trauma, neurologic disorder, genetic disorder, myocardial infarction, surgery, muscoskeletal disorder or malignancy. Patients with familial history of pain are also preferred candidates for preventive regimens. As used herein, unless otherwise indicated, the term “managing pain” encompasses preventing the recurrence of pain in a patient who has suffered from pain, and/or lengthening the time that a patient who has suffered from pain remains in remission. As used herein, unless otherwise specified, the term “modifying pain” means changing the way that a patient responds to pain. In one embodiment, “modifying pain” means bringing a patient's pain threshold from an elevated level (i.e., a level at which a patient experiences greater than normal pain in response to a particular stimulus) back to a normal level. In another embodiment, “modifying pain” means reducing a patient's pain response to a stimulus of a particular intensity. In another embodiment, modifying pain” means increasing a patient's pain threshold relative to the patient's pain threshold prior to the administration of an effective amount of a JNK Inhibitor.

### 4. DETAILED DESCRIPTION OF THE INVENTION

4.1 Illustrative JNK Inhibitors

As mentioned above, the present invention is directed to methods useful for treating, preventing, managing and/or modifying pain, comprising administering an effective amount of a JNK Inhibitor to a patient in need thereof. Illustrative JNK Inhibitors are set forth below.

In one embodiment, the JNK Inhibitor has the following structure (I):

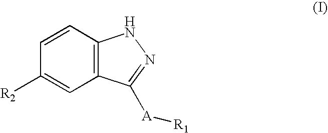

wherein:

A is a direct bond, —(CH₂)ₐ—, —(CH₂)_{b}CH═CH(CH₂)_{c}—, or —(CH₂)_{b}C═C(CH₂)_{c}—;

R₁ is aryl, heteroaryl or heterocycle fused to phenyl, each being optionally substituted with one to four substituents independently selected from R₃;

R₂ is —R₃, —R₄, —(CH₂)_{b}C(═O)R₅, —(CH₂)_{b}C(═O)OR₅, —(CH₂)_{b}C(═O)NR₅R₆, —(CH₂)_{b}C(═O)NR₅(CH₂)_{c}—(═O)R₆, —(CH₂)_{b}NR₅C(═O)R₆, —(CH₂)_{b}NR₅C(═O)NR₆R₇, —(CH₂)_{b}NR₅R₆, —(CH₂)_{b}OR₅, —(CH₂)_{b}SO_{d}R₅ or —(CH₂)_{b}SO₂NR₅R₆;

a is 1, 2, 3, 4, 5 or 6;

b and c are the same or different and at each occurrence independently selected from 0, 1, 2, 3 or 4;

d is at each occurrence 0, 1 or 2;

R₃ is at each occurrence independently halogen, hydroxy, carboxy, alkyl, alkoxy, haloalkyl, acyloxy, thioalkyl, sulfinylalkyl, sulfonylalkyl, hydroxyalkyl, aryl, arylalkyl, heterocycle, heterocycloalkyl, —C(═O)OR₈, —OC(═O)R₈, —C(═O)NR₈R₉, —C(═O)NR₈OR₉, —SO₂NR₈R₉, —NR₈SO₂R₉, —CN, —NO₂, —NR₈R₉, —NR₈C(═O)R₉, —NR₈C(═O)(CH₂)_{b}OR₉, —NR₈C(═O)(CH₂)_{b}R₉, —O(CH₂)_{b}NR₈R₉, or heterocycle fused to phenyl;

R₄ is alkyl, aryl, arylalkyl, heterocycle or heterocycloalkyl, each being optionally substituted with one to four substituents independently selected from R₃, or R₄ is halogen or hydroxy;

R₅, R₆ and R₇ are the same or different and at each occurrence independently hydrogen, alkyl, aryl, arylalkyl, heterocycle or heterocycloalkyl, wherein each of R₅, R₆ and R₇ are optionally substituted with one to four substituents independently selected from R₃; and

R₈ and R₉ are the same or different and at each occurrence independently hydrogen, alkyl, aryl, arylalkyl, heterocycle, or heterocycloalkyl, or R₈ and R₉ taken together with the atom or atoms to which they are bonded form a heterocycle, wherein each of R₈, R₉, and R₈ and R₉ taken together to form a heterocycle are optionally substituted with one to four substituents independently selected from R₃.

In one embodiment, -A-R₁ is phenyl, optionally substituted with one to four substituents independently selected from halogen, alkoxy, —NR₈C(═O)R₉, —C(═O)NR₈R₉, and —O(CH₂)_{b}NR₈R₉, wherein b is 2 or 3 and wherein R₈ and R₉ are defined above.

In another embodiment, R₂ is —R₄, —(CH₂)_{b}C(═O)R₅, —(CH₂)_{b}C(═O)OR₅, —(CH₂)_{b}C(═O)NR₅R₆, —(CH₂)_{b}C(═O)NR₅(CH₂)_{c}C(═O)R₆, —(CH₂)_{b}NR₅C(═O)R₆, —(CH₂)_{b}NR₅C(═O)NR₆R₇, —(CH₂)_{b}NR₅R₆, —(CH₂)_{b}OR₅, —(CH₂)_{b}SO_{d}R₅ or —(CH₂)_{b}SO₂NR₅R₆, and b is an integer ranging from 0-4.

In another embodiment, R₂ is —(CH₂)_{b}C(═O)NR₅R₆, —(CH₂)_{b}NR₅C(═O)R₆, 3-triazolyl or 5-tetrazolyl, wherein b is 0 and wherein R₈ and R₉ are defined above.

In another embodiment, R₂ is 3-triazolyl or 5-tetrazolyl.

In another embodiment:

(a)-A-R₁ is phenyl, optionally substituted with one to four substituents independently selected from halogen, alkoxy, —NR₈C(═O)R₉, —C(═O)NR₈R₉,

and —O(CH₂)_{b}NR₈R₉, wherein b is 2 or 3; and

(b) R₂ is —(CH₂)_{b}C(═O)NR₅R₆, —(CH₂)_{b}NR₅C(═O)R_{6,3}-triazolyl or 5-tetrazolyl, wherein b is 0 and wherein R₈ and R₉ are defined above.

In another embodiment:

(a)-A-R₁ is phenyl, optionally substituted with one to four substituents independently selected from halogen, alkoxy, —NR₈C(═O)R₉, —C(═O)NR₈R₉, and —O(CH₂)_{b}NR₈R₉, wherein b is 2 or 3; and

(b) R₂ is 3-triazolyl or 5-tetrazolyl.

In another embodiment, R₂ is R₄, and R₄ is 3-triazolyl, optionally substituted at its 5-position with:

(a) a C₁-C₄ straight or branched chain alkyl group optionally substituted with a hydroxyl, methylamino, dimethylamino or 1-pyrrolidinyl group; or

(b) a 2-pyrrolidinyl group.

In another embodiment, R₂ is R₄, and R₄ is 3-triazolyl, optionally substituted at its 5-position with: methyl, n-propyl, isopropyl, 1-hydroxyethyl, 3-hydroxypropyl, methylaminomethyl, dimethylaminomethyl, 1-(dimethylamino)ethyl, 1-pyrrolidinylmethyl or 2-pyrrolidinyl.

In another embodiment, the compounds of structure (I) have structure (IA) when A is a direct bond, or have structure (IB) when A is —(CH₂)ₐ—:

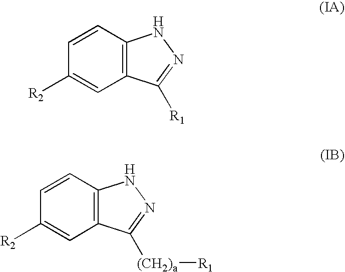

In other embodiments, the compounds of structure (I) have structure (IC) when A is a —CH₂)_{b}CH═CH(CH₂)_{c}—, and have structure (ID) when A is —(CH₂)_{b}C═C(CH₂)_{c}—:

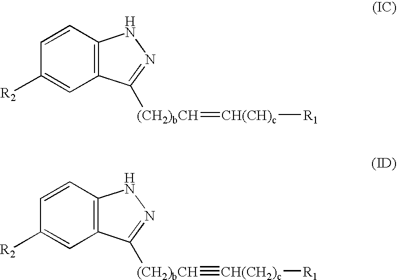

In further embodiments of this invention, R₁ of structure (I) is aryl or substituted aryl, such as phenyl or substituted phenyl as represented by the following structure (IE):

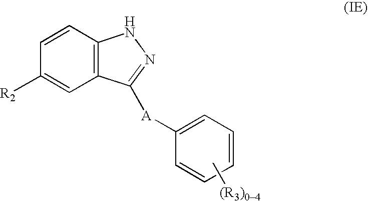

In another embodiment, R₂ of structure (I) is —(CH₂)_{b}NR₄(C═O)R₅. In one aspect of this embodiment, b=0 and the compounds have the following structure (IF):

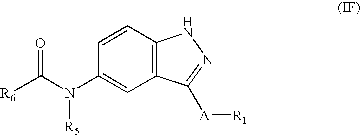

Representative R₂ groups of the compounds of structure (I) include alkyl (such as methyl and ethyl), halo (such as chloro and fluoro), haloalkyl (such as trifluoromethyl), hydroxy, alkoxy (such as methoxy and ethoxy), amino, arylalkyloxy (such as benzyloxy), mono- or di-alkylamine (such as —NHCH₃, —N(CH₃)₂ and —NHCH₂CH₃), —NHC(═O)R₄ wherein R₆ is a substituted or unsubstituted phenyl or heteroaryl (such as phenyl or heteroaryl substituted with hydroxy, carboxy, amino, ester, alkoxy, alkyl, aryl, haloalkyl, halo, —CONH₂ and —CONH alkyl), —NH(heteroarylalkyl) (such as —NHCH₂(3-pyridyl), —NHCH₂(4-pyridyl), heteroaryl (such as pyrazolo, triazolo and tetrazolo), —C(═O)NHR₆ wherein R₆ is hydrogen, alkyl, or as defined above (such as —C(═O)NH₂, —C(═O)NHCH₃, —C(═O)NH(H-carboxyphenyl), —C(═O)N(CH₃)₂), arylalkenyl (such as phenylvinyl, 3-nitrophenylvinyl, 4-carboxyphenylvinyl), heteroarylalkenyl (such as 2-pyridylvinyl, 4-pyridylvinyl).

Representative R₃ groups of the compounds of structure (I) include halogen (such as chloro and fluoro), alkyl (such as methyl, ethyl and isopropyl), haloalkyl (such as trifluoromethyl), hydroxy, alkoxy (such as methoxy, ethoxy, n-propyloxy and isobutyloxy), amino, mono- or di-alkylamino (such as dimethylamine), aryl (such as phenyl), carboxy, nitro, cyano, sulfinylalkyl (such as methylsulfinyl), sulfonylalkyl (such as methylsulfonyl), sulfonamidoalkyl (such as —NHSO₂CH₃), —NR₈C(═O)(CH₂)_{b}OR₉ (such as NHC(═O)CH₂OCH₃), NHC(═O)R₉ (such as —NHC(═O)CH₃, —NHC(═O)CH₂C₆H₅, —NHC(═O)(2-furanyl)), and —O(CH₂)_{b}NR₈R₉ (such as —O(CH₂)₂N(CH₃)₂).

The compounds of structure (I) can be made using organic synthesis techniques known to those skilled in the art, as well as by the methods described in International Publication No. WO 02/10137 (particularly in Examples 1-430, at page 35, line 1 to page 396, line 12), published Feb. 7, 2002, which is incorporated herein by reference in its entirety. Further, specific examples of these compounds are found in this publication.

Illustrative examples of JNK Inhibitors of structure (1) are:

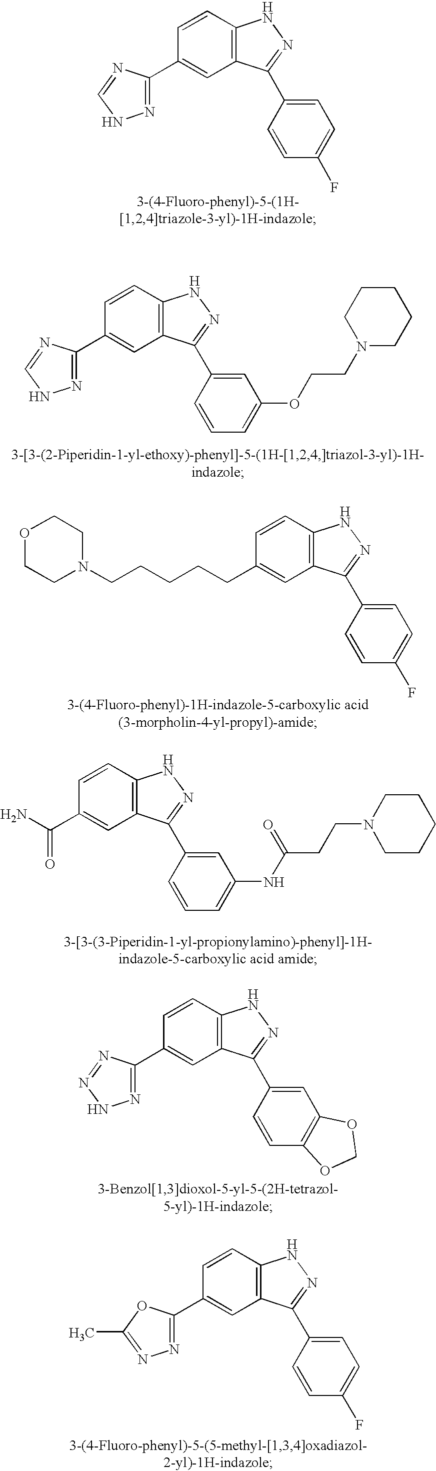

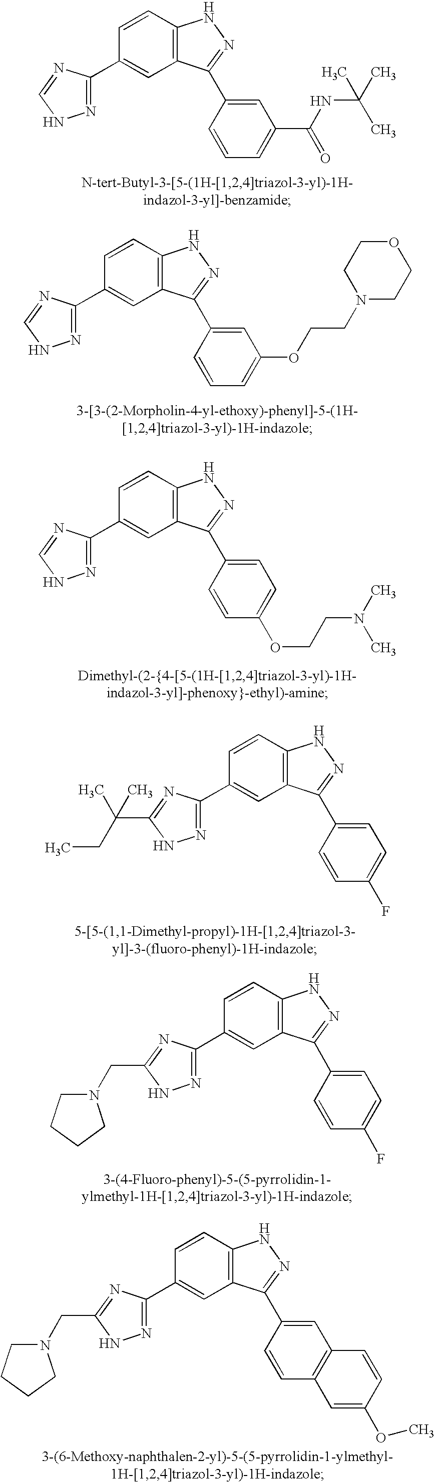

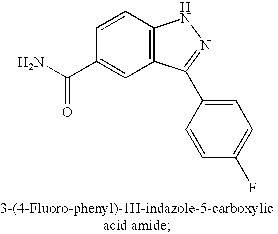

and pharmaceutically acceptable salts thereof.

In another embodiment, the JNK Inhibitor has the following structure (II):

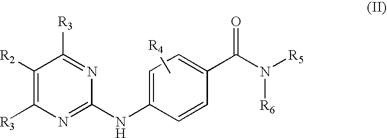

wherein:

R₁ is aryl or heteroaryl optionally substituted with one to four substituents independently selected from R₇;

R₂ is hydrogen;

R₃ is hydrogen or lower alkyl;

R₄ represents one to four optional substituents, wherein each substituent is the same or different and independently selected from halogen, hydroxy, lower alkyl and lower alkoxy;

R₅ and R₆ are the same or different and independently —R₈, —(CH₂)ₐC(═O)R₉, —(CH₂)ₐC(═O)OR₉, —(CH₂)ₐC(═O)NR₉R₁₀, —(CH₂)ₐC(═O)NR₉(CH₂)_{b}C(═O)R₁₀, —(CH₂)ₐNR₉C(═O)R₁₀, (CH₂)ₐNR₁₁C(═O)NR₉R₁₀, —(CH₂)ₐNR₉R₁₀, —(CH₂)ₐOR₉, —(CH₂)ₐSO_{c}R₉ or —(CH₂)ₐSO₂NR₉R₁₀;

or R₅ and R₆ taken together with the nitrogen atom to which they are attached to form a heterocycle or substituted heterocycle;

R₇ is at each occurrence independently halogen, hydroxy, cyano, nitro, carboxy, alkyl, alkoxy, haloalkyl, acyloxy, thioalkyl, sulfinylalkyl, sulfonylalkyl, hydroxyalkyl, aryl, arylalkyl, heterocycle, substituted heterocycle, heterocycloalkyl, —C(═O)OR₈, —OC(═O)R₈, —C(═O)NR₈R₉, —C(═O)NR₈OR₉, —SO_{c}R₈, —SO_{c}NR₈R₉, —NR₈SO_{c}R₉, —NR₈R₉, —NR₈C(═O)R₉, —NR₈C(═O)(CH₂)_{b}OR₉, —NR₈C(═O)(CH₂)_{b}R₉, —O(CH₂)_{b}NR₈R₉, or heterocycle fused to phenyl;

R₉, R₉, R₁₀ and R₁₁ are the same or different and at each occurrence independently hydrogen, alkyl, aryl, arylalkyl, heterocycle, heterocycloalkyl;

or R₈ and R₉ taken together with the atom or atoms to which they are attached to form a heterocycle;

a and b are the same or different and at each occurrence independently selected from 0, 1, 2, 3 or 4; and

c is at each occurrence 0, 1 or 2.

In one embodiment, R₁ is a substituted or unsubstituted aryl or heteroaryl. When R₁ is substituted, it is substituted with one or more substituents defined below. In one embodiment, when substituted, R₁ is substituted with a halogen, —SO₂R₈ or —SO₂R₈R₉.

In another embodiment, R₁ is substituted or unsubstituted aryl, furyl, benzofuranyl, thiophenyl, benzothiophenyl, quinolinyl, pyrrolyl, indolyl, oxazolyl, benzoxazolyl, imidazolyl, benzimidazolyl, thiazolyl, benzothiazolyl, isoxazolyl, pyrazolyl, isothiazolyl, pyridazinyl, pyrimidinyl, pyrazinyl, triazinyl, cinnolinyl, phthalazinyl or quinazolinyl.

In another embodiment R is substituted or unsubstituted aryl or heteroaryl. When R₁ is substituted, it is substituted with one or more substituents defined below. In one embodiment, when substituted, R₁ is substituted with a halogen, —SO₂R₈ or —SO₂R₈R₉.

In another embodiment, R₁ is substituted or unsubstituted aryl, preferably phenyl. When R₁ is a substituted aryl, the substituents are defined below. In one embodiment, when substituted, R₁ is substituted with a halogen, —SO₂R₈ or —SO₂R₈R₉.

In another embodiment, R₅ and R₆, taken together with the nitrogen atom to which they are attached form a substituted or unsubstituted nitrogen-containing non-aromatic heterocycle, in one embodiment, piperazinyl, piperidinyl or morpholinyl.

When R₅ and R₆, taken together with the nitrogen atom to which they areattached form substituted piperazinyl, piperadinyl or morpholinyl, the piperazinyl, piperadinyl or morpholinyl is substituted with one or more substituents defined below. In one embodiment, when substituted, the substituent is alkyl, amino, alkylamino, alkoxyalkyl, acyl, pyrrolidinyl or piperidinyl.

In one embodiment, R₃ is hydrogen and R₄ is not present, and the JNK Inhibitor has the following structure (IIA):

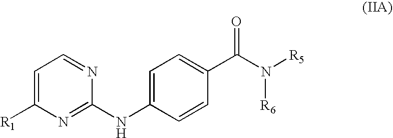

and pharmaceutically acceptable salts thereof.

In a more specific embodiment, R₁ is phenyl optionally substituted with R₇, and having the following structure (IIB):

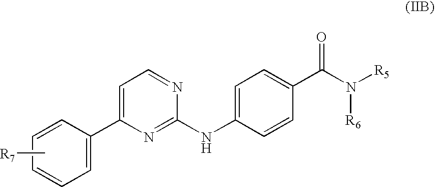

and pharmaceutically acceptable salts thereof.

In still a further embodiment, R₇ is at the para position of the phenyl group relative to the pyrimidine, as represented by the following structure (IIC):

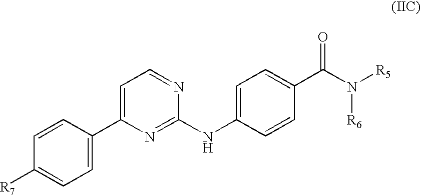

and pharmaceutically acceptable salts thereof.

The JNK Inhibitors of structure (II) can be made using organic synthesis techniques known to those skilled in the art, as well as by the methods described in International Publication No. WO 02/46170 (particularly Examples 1-27 at page 23, line 5 to page 183, line 25), published Jun. 13, 2002, which is hereby incorporated by reference in itsr entirety. Further, specific examples of these compounds are found in the publication.

Illustrative examples of JNK Inhibitors of structure (II) are:

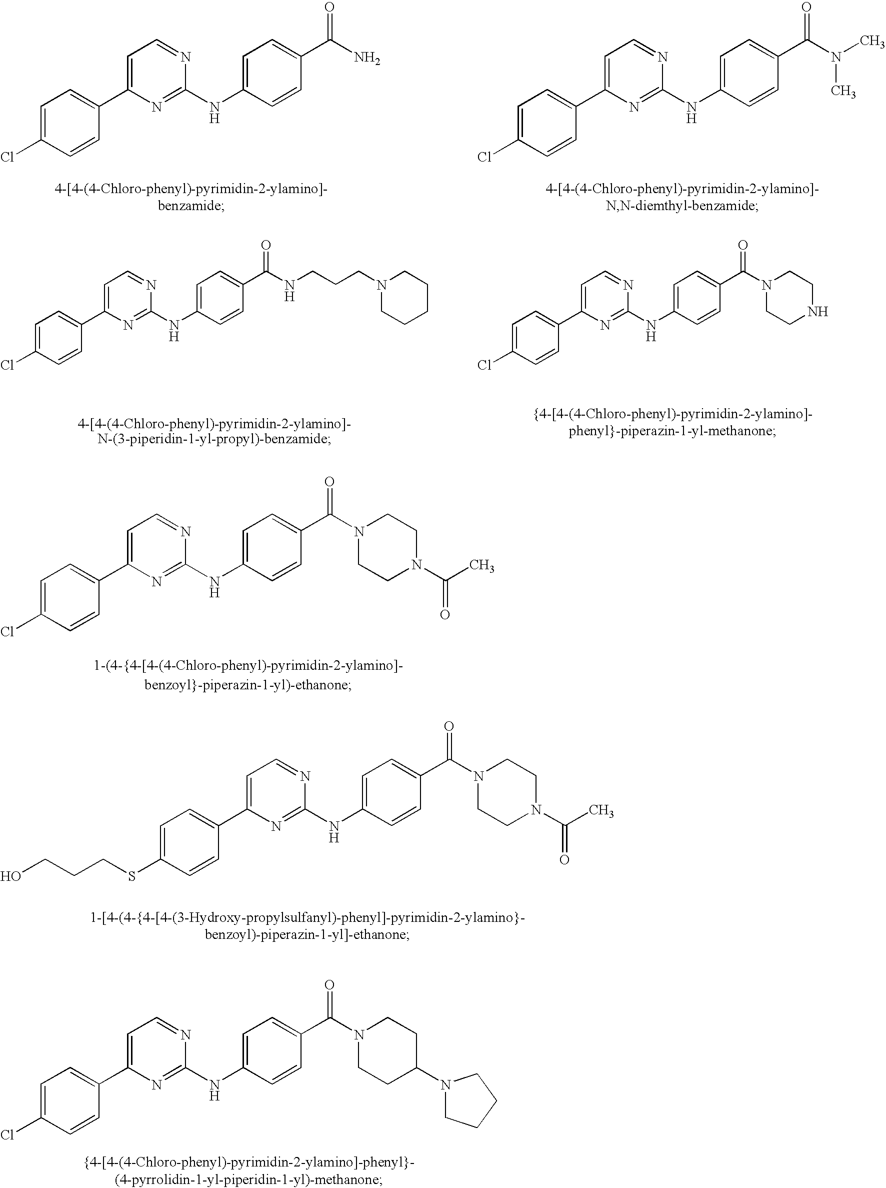

and pharmaceutically acceptable salts thereof.

In another embodiment, the JNK Inhibitor has the following structure (III):

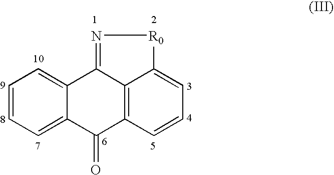

wherein R₀ is —O—, —S—, —S(O)—, —S(O)₂—, NH or —CH₂—;

the compound of structure (III) being: (i) unsubstituted, (ii) monosubstituted and having a first substituent, or (iii) disubstituted and having a first substituent and a second substituent;

the first or second substituent, when present, is at the 3, 4, 5, 7, 8, 9, or 10 position, wherein the first and second substituent, when present, are independently alkyl, hydroxy, halogen, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono-alkylaminoalkoxy, di-alkylaminoalkoxy, or a group represented by structure (a), (b), (c), (d), (e), or (f):

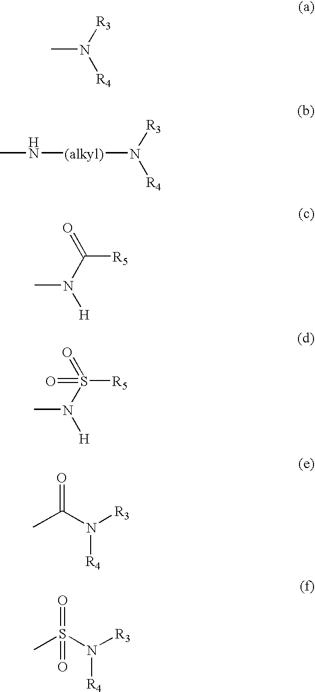

wherein R₃ and R₄ are taken together and represent alkylidene or a heteroatom-containing cyclic alkylidene or R₃ and R₄ are independently hydrogen, alkyl, cycloalkyl, aryl, arylalkyl, cycloalkylalkyl, aryloxyalkyl, alkoxyalkyl, aminoalkyl, mono-alkylaminoalkyl, or di-alkylaminoalkyl; and

R₅ is hydrogen, alkyl, cycloalkyl, aryl, arylalkyl, cycloalkylalkyl, alkoxy, alkoxyalkyl, alkoxycarbonylalkyl, amino, mono-alkylamino, di-alkylamino, arylamino, arylalkylamino, cycloalkylamino, cycloalkylalkylamino, aminoalkyl, monoalkylaminoalkyl, or di-alkylaminoalkyl.

In another embodiment, the JNK Inhibitor has the following structure (IIA):

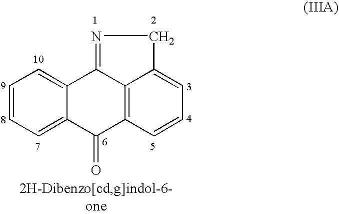

being: (i) unsubstituted, (ii) monosubstituted and having a first substituent, or (iii) disubstituted and having a first substituent and a second substituent;

the first or second substituent, when present, is at the 3, 4, 5, 7, 8, 9, or 10 position;

wherein the first and second substituent, when present, are independently alkyl, hydroxy, halogen, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono-alkylaminoalkoxy, di-alkylaminoalkoxy, or a group represented by structure (a), (b), (c), (d), (e), or (f):

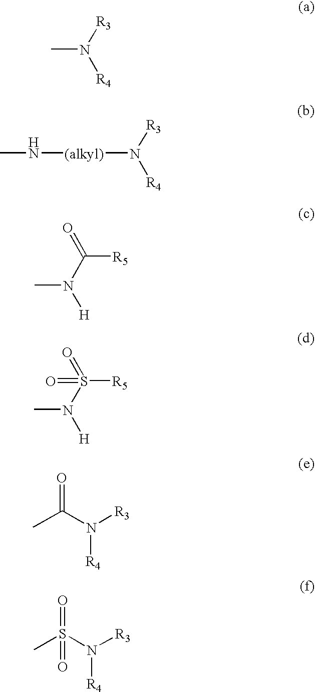

wherein R₃ and R₄ are taken together and represent alkylidene or a heteroatom-containing cyclic alkylidene or R₃ and R₄ are independently hydrogen, alkyl, cycloalkyl, aryl, arylalkyl, cycloalkylalkyl, aryloxyalkyl, alkoxyalkyl, aminoalkyl, mono-alkylaminoalkyl, or di-alkylaminoalkyl; and

R₅ is hydrogen, alkyl, cycloalkyl, aryl, arylalkyl, cycloalkylalkyl, alkoxy, alkoxyalkyl, alkoxycarbonylalkyl, amino, mono-alkylamino, di-alkylamino, arylamino, arylalkylamino, cycloalkylamino, cycloalkylalkylamino, aminoalkyl, monoalkylaminoalkyl, or di-alkylaminoalkyl.

A subclass of the compounds of structure (IIIA) is that wherein the first or second substituent is present at the 5, 7, or 9 position. In one embodiment, the first or second substituent is present at the 5 or 7 position.

A second subclass of compounds of structure (IIIA) is that wherein the first or second substituent is present at the 5, 7, or 9 position;

the first or second substituent is independently alkoxy, aryloxy, aminoalkyl, mono-alkylaminoalkyl, di-alkylaminoalkyl, or a group represented by the structure (a), (c), (d), (e), or (f);

R₃ and R₄ are independently hydrogen, alkyl, cycloalkyl, aryl, arylalkyl, or cycloalkylalkyl; and

R₅ is hydrogen, alkyl, cycloalkyl, aryl, arylalkyl, or cycloalkylalkyl.

In another embodiment, the JNK Inhibitor has the following structure (IIIB):

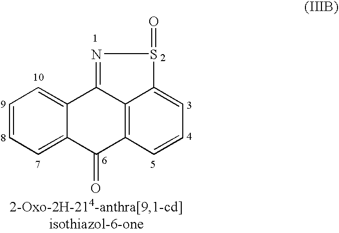

being (i) unsubstituted, (ii) monosubstituted and having a first substituent, or (ii) disubstituted and having a first substituent and a second substituent;

the first or second substituent, when present, is at the 3, 4, 5, 7, 8, 9, or 10 position;

wherein the first and second substituent, when present, are independently alkyl, halogen, hydroxy, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono-alkylaminoalkoxy, di-alkylaminoalkoxy, or a group represented by structure (a), (b) (c), (d), (e), or (f):

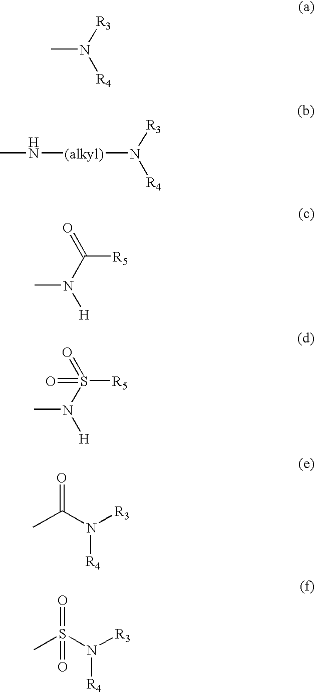

wherein R₃ and R₄ are taken together and represent alkylidene or a heteroatom-containing cyclic alkylidene or R₃ and R₄ are independently hydrogen, alkyl, cycloalkyl, aryl, arylalkyl, cycloalkylalkyl, aryloxyalkyl, alkoxyalkyl, aminoalkyl, mono-alkylaminoalkyl, or di-alkylaminoalkyl; and

R₅ is hydrogen, alkyl, cycloalkyl, aryl, arylalkyl, cycloalkylalkyl, alkoxy, alkoxyalkyl, alkoxycarbonylalkyl, amino, mono-alkylamino, di-alkylamino, arylamino, arylalkylamino, cycloalkylamino, cycloalkylalkylamino, aminoalkyl, mono-alkylaminoalkyl, or di-alkylaminoalkyl.

A subclass of the compounds of structure (IIIB) is that wherein the first or second substituent is present at the 5, 7, or 9 position. In one embodiment, the first or second substituent is present at the 5 or 7 position.

A second subclass of the compounds of structure (IIIB) is that wherein the first or second substituent is independently alkoxy, aryloxy, or a group represented by the structure (a), (c), (d), (e), or (f);

R₃ and R₄ are independently hydrogen, alkyl, cycloalkyl, aryl, arylalkyl, or cycloalkylalkyl; and

R₅ is hydrogen, alkyl, cycloalkyl, aryl, arylatkyl, or cycloalkylalkyl.

In another embodiment, the JNK Inhibitor has the following structure (IIIC):

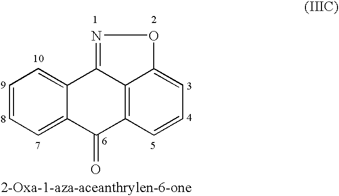

being (i) monosubstituted and having a first substituent or (ii) disubstituted and having a first substituent and a second substituent;

the first or second substituent, when present, is at the 3, 4, 5, 7, 8, 9, or 10 position;

wherein the first and second substituent, when present, are independently alkyl, halogen, hydroxy, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono-alkylaminoalkoxy, di-alkylaminoalkoxy, or a group represented by structure (a), (b), (c) (d), (e), or (f):

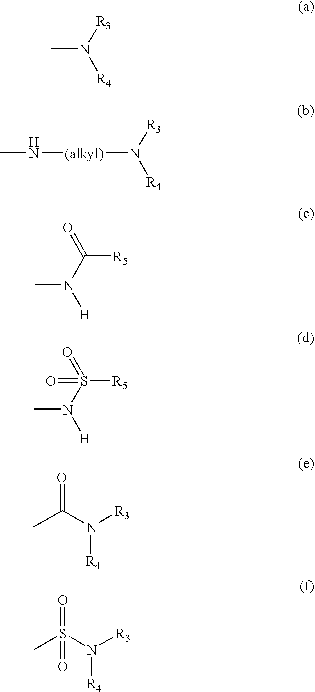

wherein R₃ and R₄ are taken together and represent alkylidene or a heteroatom-containing cyclic alkylidene or R₃ and R₄ are independently hydrogen, alkyl, cycloalkyl, aryl, arylalkyl, cycloalkylalkyl, aryloxyalkyl, alkoxyalkyl, aminoalkyl, mono-alkylaminoalkyl, or di-alkylaminoalkyl; and

R₅ is hydrogen, alkyl, cycloalkyl, aryl, arylalkyl, cycloalkylalkyl, alkoxy, alkoxyalkyl, alkoxycarbonylalkyl, amino, mono-alkylamino, di-alkylamino, arylamino, arylalkylamino, cycloalkylamino, cycloalkylalkylamino, aminoalkyl, monoalkylaminoalkyl, or di-alkylaminoalkyl.

A subclass of the compounds of structure (IIIC) is that wherein the first or second substituent is present at the 5, 7, or 9 position. In one embodiment, the first or second substituent is present at the 5 or 7 position.

A second subclass of the compounds of structure (IIIC) is that wherein the first or second substituent is independently alkoxy, aryloxy, aminoalkyl, mono-alkylaminoalkyl, di-alkylaminoalkyl, or a group represented by the structure (a), (c), (d), (e), or (f);

R₃ and R₄ are independently hydrogen, alkyl, cycloalkyl, aryl, arylalkyl, or cycloalkylalkyl; and

R₅ is hydrogen, alkyl, cycloalkyl, aryl, arylalkyl, or cycloalkylalkyl.

In another embodiment, the JNK Inhibitor has the following structure (IIID):

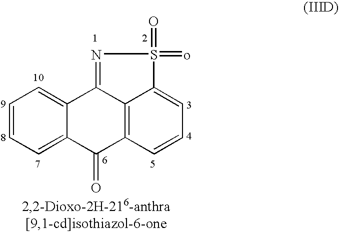

being (i) monosubstituted and having a first substituent present at the 5, 7, or 9 position, (ii) disubstituted and having a first substituent present at the 5 position and a second substituent present at the 7 position, (iii) disubstituted and having a first substituent present at the 5 position and a second substituent present at the 9 position, or (iv) disubstituted and having a first substituent present at the 7 position and a second substituent present at the 9 position;

wherein the first and second substituent, when present, are independently alkyl, halogen, hydroxy, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono-alkylaminoalkoxy, di-alkylaminoalkoxy, or a group represented by structure (a), (b), (c), (d), (e), or (f):

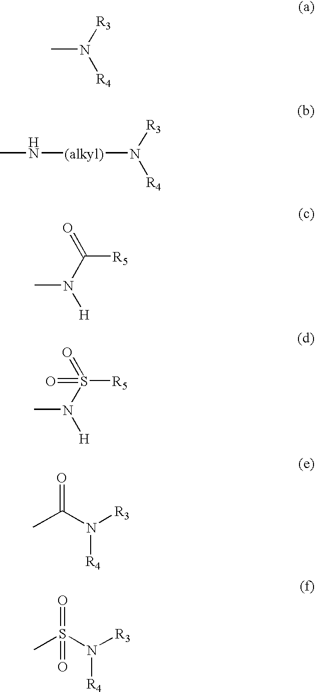

wherein R₃ and R₄ are taken together and represent alkylidene or a heteroatom-containing cyclic alkylidene or R₃ and R₄ are independently hydrogen, alkyl, cycloalkyl, aryl, arylalkyl, cycloalkylalkyl, aryloxyalkyl, alkoxyalkyl, aminoalkyl, mono-alkylaminoalkyl, or di-alkylaminoalkyl; and

R₅ is hydrogen, alkyl, cycloalkyl, aryl, arylalkyl, cycloalkylalkyl, alkoxy, alkoxyalkyl, alkoxycarbonylalkyl, amino, mono-alkylamino, di-alkylamino, arylamino, arylalkylamino, cycloalkylamino, cycloalkylalkylamino, aminoalkyl, monoalkylaminoalkyl, or di-alkylaminoalkyl.

A subclass of the compounds of structure (IIID) is that wherein the first or second substituent is present at the 5 or 7 position.

A second subclass of the compounds of structure (IIID) is that wherein the first or second substituent is independently alkyl, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono-alkylaminoalkoxy, di-alkylaminoalkoxy, or a group represented by structure (a), (c), (d), (e), or (f).

Another subclass of the compounds of structure (IIID) is that wherein the first and second substituent are independently alkoxy, aryloxy, or a group represented by the structure (a), (c), (d), (e), or (f);

R₃ and R₄ are independently hydrogen, alkyl, cycloalkyl, aryl, arylalkyl, or cycloalkylalkyl; and

R₅ is hydrogen, alkyl, cycloalkyl, aryl, arylalkyl, alkoxycarbonyl, or cycloalkylalkyl.

In another embodiment, the JNK Inhibitor has the following structure (IIE):

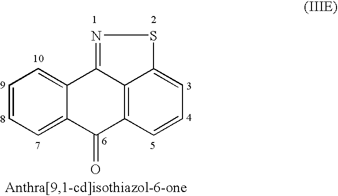

being (i) monosubstituted and having a first substituent present at the 5, 7, or 9 position, (ii) disubstituted and having a first substituent present at the 5 position and a second substituent present at the 9 position, (iii) disubstituted and having a first substituent present at the 7 position and a second substituent present at the 9 position, or (iv) disubstituted and having a first substituent present at the 5 position and a second substituent present at the 7 position;

wherein the first and second substituent, when present, are independently alkyl, halogen, hydroxy, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono-alkylaminoalkoxy, di-alkylaminoalkoxy, or a group represented by structure (a), (b), (c), (d), (e), or (f):

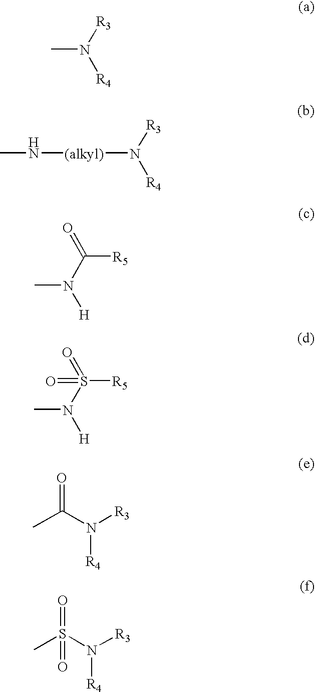

wherein R₃ and R₄ are taken together and represent alkylidene or a heteroatom-containing cyclic alkylidene or R₃ and R₄ are independently hydrogen, alkyl, cycloalkyl, aryl, arylalkyl, cycloalkylalkyl, aryloxyalkyl, alkoxyalkyl, aminoalkyl, mono-alkylaminoalkyl, or di-alkylaminoalkyl; and

R₅ is hydrogen, alkyl, cycloalkyl, aryl, arylalkyl, cycloalkylalkyl, alkoxy, alkoxyalkyl, alkoxycarbonylalkyl, amino, mono-alkylamino, di-alkylamino, arylamino, arylalkylamino, cycloalkylamino, cycloalkylalkylamino, aminoalkyl, monoalkylaminoalkyl, or di-alkylaminoalkyl.

A subclass of the compounds of structure (IIIE) is that wherein the first or second substituent is present at the 5 or 7 position.

A second subclass of the compounds of structure (IIIE) is that wherein the compound of structure (IIIE) is disubstituted and at least one of the substituents is a group represented by the structure (d) or (f).

Another subclass of the compounds of structure (IIIE) is that wherein the compounds are monosubstituted. Yet another subclass of compounds is that wherein the compounds are monosubstituted at the 5 or 7 position with a group represented by the structure (e) or (f).

In another embodiment, the JNK Inhibitor has the following structure (IIIF):

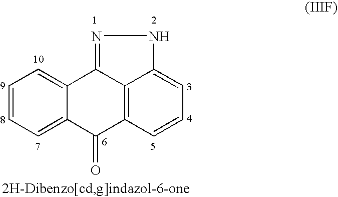

being (i) unsubstituted, (ii) monosubstituted and having a first substituent, or (iii) disubstituted and having a first substituent and a second substituent;

the first or second substituent, when present, is at the 3, 4, 5, 7, 8, 9, or 10 position;

wherein the first and second substituent, when present, are independently alkyl, hydroxy, halogen, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono-alkylaminoalkoxy, dialkylaminoalkoxy, or a group represented by structure (a), (b), (c), (d), (e), or (f):

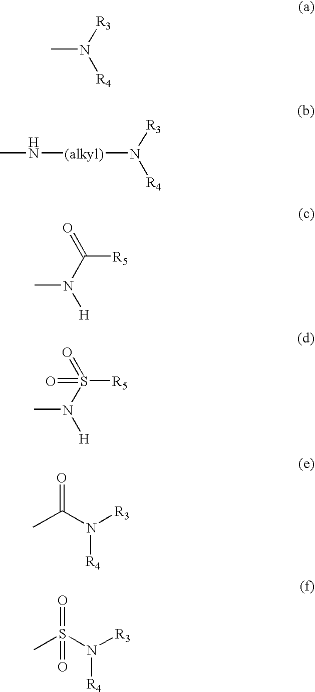

wherein R₃ and R₄ are taken together and represent alkylidene or a heteroatom-containing cyclic alkylidene or R₃ and R₄ are independently hydrogen, alkyl, cycloalkyl, aryl, arylalkyl, cycloalkylalkyl, aryloxyalkyl, alkoxyalkyl, aminoalkyl, mono-alkylaminoalkyl, or di-alkylaminoalkyl; and

R₅ is hydrogen, alkyl, cycloalkyl, aryl, arylalkyl, cycloalkylalkyl, alkoxy, alkoxyalkyl, alkoxycarbonylalkyl, amino, mono-alkylamino, di-alkylamino, arylamino, arylalkylamino, cycloalkylamino, cycloalkylalkylamino, aminoalkyl, mono-alkylaminoalkyl, or di-alkylaminoalkyl.

In one embodiment, the compound of structure (IIIF), or a pharmaceutically acceptable salt thereof is unsubstituted at the 3, 4, 5, 7, 8, 9, or 10 position.

The JNK Inhibitors of structure (III) can be made using organic synthesis techniques known to those skilled in the art, as well as by the methods described in International Publication No. WO 01/12609 (particularly Examples 1-7 at page 24, line 6 to page 49, line 16), published Feb. 22, 2001, as well as International Publication No. WO 02/066450 (particularly compounds AA-HG at pages 59-108), published Aug. 29, 2002, each of which is hereby incorporated by reference in its entirety. Further, specific examples of these compounds can be found in the publications.

Illustrative examples of JNK Inhibitors of structure (III) are:

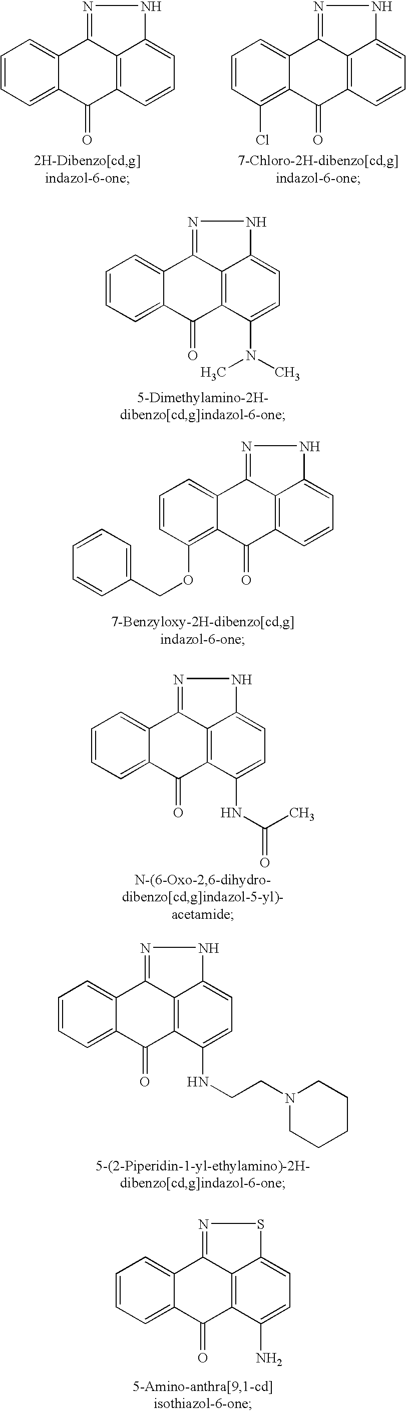

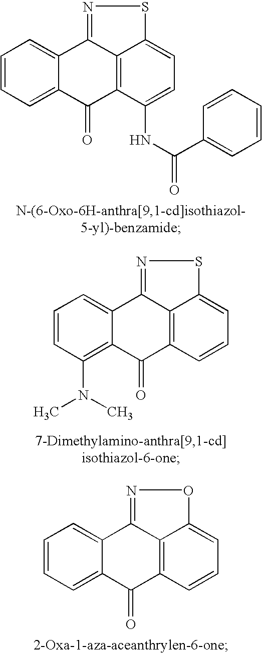

and pharmaceutically acceptable salts thereof.

Other JNK Inhibitors that are useful in the present methods include, but are not limited to, those disclosed in International Publication No. WO 00/39101, (particularly at page 2, line 10 to page 6, line 12); International Publication No. WO 01/14375 (particularly at page 2, line 4 to page 4, line 4); International Publication No. WO 00/56738 (particularly at page 3, line 25 to page 6, line 13); International Publication No. WO 01/27089 (particularly at page 3, line 7 to page 5, line 29); International Publication No. WO 00/12468 (particularly at page 2, line 10 to page 4, line 14); European Patent Publication 1 110 957 (particularly at page 19, line 52 to page 21, line 9); International Publication No. WO 00/75118 (particularly at page 8, line 10 to page 11, line 26); International Publication No. WO 01/12621 (particularly at page 8, line 10 to page 10, line 7); International Publication No. WO 00/64872 (particularly at page 9, line 1 to page, 106, line 2); International Publication No. WO 01/23378 (particularly at page 90, line 1 to page 91, linel 1); International Publication No. WO 02/16359 (particularly at page 163, line 1 to page 164, line 25); U.S. Pat. No. 6,288,089 (particularly at column 22, line 25 to column 25, line 35); U.S. Pat. No. 6,307,056 (particularly at column 63, line 29 to column 66, line 12); International Publication No. WO 00/35921 (particularly at page 23, line 5 to page 26, line 14); International Publication No. WO 01/91749 (particularly at page 29, lines 1-22); International Publication No. WO 01/56993 (particularly in at page 43 to page 45); and International Publication No. WO 01/58448 (particularly in at page 39), each of which is incorporated by reference herein in its entirety.

Pharmaceutical compositions including dosage forms of the invention, which comprise an effective amount of a JNK Inhibitor can be used in the methods of the invention.

4.2 Methods of Use

This invention is based, in part, on the belief that a JNK Inhibitor can work alone or in combination with another active agent or physical therapy to effectively treat, prevent, manage and/or modify varying types and severities of pain. Without being limited by theory, compounds of the invention can, but do not necessarily, act as analgesics. In particular, because a JNK Inhibitor can dramatically affect the production of cytokines (e.g., TNF-α), it is believed that they can function as “antihyperalgesics” and/or “neuromodulators” by restoring the baseline or normal pain threshold of the injured patient to which they are administered. Thus, a JNK Inhibitor can act differently than an analgesic, which typically diminishes the response induced by stimulus, by instead altering the patient's ability to withstand that response either by suppressing the suffering associated with the pain or directly reducing the responsiveness of the nociceptors. For this reason, it is believed that a JNK Inhibitor can be used to treat, prevent, manage and/or modify not only nociceptive pain, but other types of pain (e.g., neuropathic pain) with substantially different etiologies. Moreover, because of the unique mechanism by which a JNK Inhibitor is believed to act, it is believed that a JNK Inhibitor can relieve or reduce pain without incurring adverse effects (e.g., narcotic effects) typical of some analgesics (e.g., opioids), even when administered systemically.

Methods of this invention encompass methods for treating, preventing, managing and/or modifying various types of pain and related syndromes, comprising administering an effective amount of a JNK Inhibitor to a patient in need thereof.

In one embodiment, the invention relates to a method for treating, preventing, managing and/or modifying nociceptive pain, comprising administering an effective amount of a JNK Inhibitor to a patient in need thereof. In certain embodiments, the nociceptive pain results from physical trauma (e.g., a cut or contusion of the skin; or a chemical or thermal burn), osteoarthritis, rheumatoid arthritis or tendonitis. In another embodiment, the nociceptive pain is myofascial pain.

In another embodiment, the invention relates to a method for treating, preventing, managing and/or modifying neuropathic pain, comprising administering an effective amount of a JNK Inhibitor to a patient in need thereof. In certain embodiments, the neuropathic pain is associated with stroke, diabetic neuropathy, luetic neuropathy, postherpetic neuralgia, trigeminal neuralgia, fibromyalgia, or painful neuropathy induced iatrogenically by drugs such as vincristine, velcade or thalidomide.

In another embodiment, the invention relates to a method for treating, preventing, managing and/or modifying mixed pain (i.e., pain with both nociceptive and neuropathic components), comprising administering an effective amount of a JNK Inhibitor to a patient in need thereof.

In another embodiment, the invention relates to a method for treating, preventing, managing and/or modifying visceral pain; headache pain (e.g., migraine headache pain); mixed pain (i.e., chronic pain having nociceptive and neuropathic components); CRPS; CRPS type I; CRPS type II; RSD; reflex neurovascular dystrophy; reflex dystrophy; sympathetically maintained pain syndrome; causalgia; Sudeck atrophy of bone; algoneurodystrophy; shoulder hand syndrome; post-traumatic dystrophy; autonomic dysfunction; cancer-related pain; phantom limb pain; fibromyalgia; myofascial pain; chronic fatigue syndrome; post-operative pain; spinal cord injury pain; central post-stroke pain; radiculopathy; sensitivity to temperature, light touch or color change to the skin (allodynia); pain from hyperthermic or hypothermic conditions; and other painful conditions (e.g., diabetic neuropathy, luetic neuropathy, postherpetic neuralgia, trigeminal neuralgia, or painful neuropathy induced iatrogenically by drugs such as vincristine, velcade or thalidomide), comprising administering an effective amount of a JNK Inhibitor to a patient in need thereof.

In another embodiment, the invention relates to a method for treating, preventing, managing and/or modifying pain associated with a cytokine, comprising administering an effective amount of a JNK Inhibitor to a patient in need thereof. In one embodiment, inhibiting cytokine activity or cytokine production results in the treatment, prevention, management and/or modification of the pain. In another embodiment, the cytokine is TNF-α. In another embodiment, the pain associated with a cytokine is nociceptive pain. In another embodiment, the pain associated with a cytokine is neuropathic pain.

In another embodiment, the invention relates to a method for treating, preventing, managing and/or modifying pain associated with a mitogen-activated protein kinase (MAPK), comprising administering an effective amount of a JNK Inhibitor to a patient in need thereof. In one embodiment, the MAPK is JNK (e.g, JNK1, JNK2 or JNK3). In another embodiment, the MAPK is an extracellular signal-regulated kinase (ERK) (e.g., ERK1 or ERK2). In another embodiment, the MAPK is p38.

In another embodiment, the invention relates to a method for treating, preventing, managing and/or modifying pain associated with inflammation, comprising administering an effective amount of a JNK Inhibitor to a patient in need thereof.

In another embodiment, the invention relates to a method of treating, preventing, managing and/or modifying pain associated with surgery, in one embodiment planned surgery (i.e., planned trauma), comprising administering an effective amount of a JNK Inhibitor to a patient in need thereof. In this embodiment, the JNK Inhibitor can be administered before, during and/or after the planned surgery. In a particular embodiment, the patient is administered about 5 to about 25 mg/day of a JNK Inhibitor from 1-21 days prior to the planned surgery and/or about 5 to about 25 mg/day of a JNK Inhibitor from 1-21 days after the planned surgery. In another embodiment, the patient is administered about 10 mg/day of a JNK Inhibitor from 1-21 days prior to the planned surgery and/or about 10 mg/day of a JNK Inhibitor from 1-21 days after the planned surgery.

In a further embodiment, the invention relates to methods for treating a patient who has been previously treated for pain (in particular, a patient who was non-responsive to standard pain therapy), as well as a patient who has not previously been treated for pain, comprising administering an effective amount of a JNK Inhibitor to a patient in need thereof. Because a patient experiencing pain can have heterogenous clinical manifestations and varying clinical outcomes, the treatment given to a patient can vary, depending on his/her prognosis. The skilled clinician will be able to readily determine without undue experimentation specific secondary agents, types of surgery, or types of physical therapy that can be effectively used to treat an individual patient.

In a yet a further embodiment, the invention relates to methods for managing the development and duration of pain, comprising administering to a patient in need of such management an effective amount of a JNK Inhibitor.

4.2.1 Combination Therapy With A Second Active Agent

The invention further relates to methods for treating, preventing, managing and/or modifying pain, comprising administering a JNK Inhibitor in combination with a second active agent, such as a prophylactic or therapeutic agent, to a patient in need thereof.

Examples of second active agents include, but are not limited to, conventional therapeutics used to treat, prevent, manage and/or modify pain, including, but not limited to, antidepressants, anticonvulsants, antihypertensives, anxiolytics, calcium channel blockers, muscle relaxants, non-narcotic analgesics, opioid analgesics, anti-inflammatories, cox-2 inhibitors, alpha-adrenergic receptor agonists or antagonists, ketamine, anesthetics, immunomodulatory agents, immunosuppressive agents, corticosteroids, hyperbaric oxygen, anticonvulsants, NMDA antagonists, IMiDs® and SelCIDs® (Celgene Corporation, New Jersey) (e.g., those disclosed in U.S. Pat. Nos. 6,075,041; 5,877,200; 5,698,579; 5,703,098; 6,429,221; 5,736,570; 5,658,940; 5,728,845; 5,728,844; 6,262,101; 6,020,358; 5,929,117; 6,326,388; 6,281,230; 5,635,517; 5,798,368; 6,395,754; 5,955,476; 6,403,613; 6,380,239; and 6,458,810, each of which is incorporated herein by reference), or a combination thereof, and other therapeutics found, for example, in the Physician 's Desk Reference 2003.

The specific amount of the second active agent agent will depend on the specific agent used, the type of pain being treated or managed, the severity and stage of pain, and the amount(s) of a JNK Inhibitor and any optional additional active agents concurrently administered to the patient. In a particular embodiment, the second active agent is salicyclic acid acetate, celocoxib, enbrel, thalidomide, an IMiD®, a SelCID®, gabapentin, phenyloin, carbamazepine, valproic acid, morphine sulfate, hydromorphone, prednisone, griseofulvin, penthonium, alendronate, dyphenhydramide, guanethidine, ketorolac, thyrocalcitonin, dimethylsulfoxide, clonidine, bretylium, ketanserin, reserpine, droperidol, atropine, phentolamine, bupivacaine, lidocaine, acetaminophen, nortriptyline, amitriptyline, imipramine, doxepin, clomipramine, fluoxetine, sertraline, nefazodone, venlafaxine, trazodone, bupropion, mexiletine, nifedipine, propranolol, tramadol, lamotrigine, ziconotide, ketamine, dextromethorphan, benzodiazepines, baclofen, tizanidine, phenoxybenzamine or a combination thereof, or a pharmaceutically acceptable salt, solvate, hydrate, stereoisomer, clathrate, prodrug or pharmacologically active metabolite thereof.

Hydromorphone is preferably administered in an initial dose of about 2 mg orally, or about 1 mg intravenously to manage moderate to severe pain. See, e.g., Physicians' Desk Reference, 441-446 (56^{th} ed., 2002). Morphine sulphate is preferably administered in an initial dose of about 2 mg IV/SC/IM, depending on whether a patient has already taken narcotic analgesics. See, e.g., Physicians' Desk Reference, 594-595 (56 h ed., 2002). No intrinsic limit to the amount that can be given exists, as long as a patient is observed for signs of adverse effects, especially respiratory depression. Various IV doses may be used, commonly titrated until a desired effect is obtained. For patients not using long-term agents, as little as 2 mg IV/SC may be sufficient. Larger doses are typically required for patients taking long-term narcotic analgesics. Morphine sulphate are also available in oral form in immediate-release and timed-release preparations. The long-acting oral form may be administered twice per day. An immediate-release form may be needed for periods of pain break-through, with the dose dependent on previous use. Oxycodone is a long-acting form of an opioid and may be used in initial and later stages of pain. Oxycodone is preferably administered in an amount of about 10-160 mg twice a day. See, e.g., Physicians' Desk Reference, 2912-2916 (56^{th} ed., 2002). Meperidine is preferably administered in an amount of about 50-150 mg PO/IV/IM/SC every 3-4 hours. A typical pediatric dose of meperidine is 1-1.8 mg/kg (0.5-0.8 mg/lb) PO/IV/IM/SC every 3-4 hours. See, e.g., Physicians' Desk Reference, 3079-3081 (56^{th} ed., 2002). Fentanyl transdermal patch is available as a transdermal dosage form. Most patients are administered the drug in 72 hour dosing intervals; however, some patients may require dosing intervals of about 48 hours. A typical adult dose is about 25 mcg/h (10 cm²), 50 mcg/h (20 cm²), 75 mcg/h (75 cm²), or 100 mcg/h (100 cm²). See, e.g., Physicians' Desk Reference, 1786-1789 (56′ ed., 2002).

Non-narcotic analgesics and anti-inflammatories can be used to treat patients suffering from mild to moderate pain. Anti-inflammatories such as nonsteroidal anti-inflammatory druges (NSAIDs) and cox-2 inhibitors typically inhibit inflammatory reactions and pain by decreasing activity of cyclo-oxygenase, which is responsible for prostaglandin synthesis. NSAIDs may provide pain relief in the early stage of a pain syndrome. Examples of anti-inflammatories include, but are not limited to, salicyclic acid acetate, ibuprofen, ketoprofen, rofecoxib, naproxen sodium, ketorolac, and other known conventional medications. Ibuprofen can be orally administered in an amount of 400-800 mg three times a day. See, e.g., Physicians' Desk Reference, 511, 667 and 773 (56^{th} ed., 2002); Physicians' Desk Reference for Nonprescription Drugs and Dietary Supplements, 511, 667, 773 (23rd ed., 2002). Naproxen sodium may also preferably be used for relief of mild to moderate pain in an amount of about 275 mg thrice a day or about 550 mg twice a day. See, e.g., Physicians' Desk Reference, 2967-2970 (56^{th} ed., 2002). A specific cox-2 inhibitor is celocoxib.

Antidepressants, e.g., nortriptyline, may also be used in embodiments of the invention to treat patients suffering from chronic and/or neuropathic pain. Antidepressants increase the synaptic concentration of serotonin and/or norepinephrine in the CNS by inhibiting their reuptake by presynaptic neuronal membrane. Some antidepressants also have sodium channel blocking ability to reduce the firing rate of injured peripheral afferent fibers. Examples of antidepressants include, but are not limited to, nortriptyline (Pamelor®), amitriptyline (Elavil®), imipramine (Tofranil®), doxepin (Sinequan®), clomipramine (Anafranil®), fluoxetine (Prozac®), sertraline (Zoloft®), nefazodone (Serzone®), venlafaxine (Effexor®), trazodone (Desyrel®), bupropion (Wellbutrin®) and other known conventional medications. See, e.g., Physicians' Desk Reference, 329, 1417, 1831 and 3270 (57^{th} ed., 2003). The oral adult dose is typically in an amount of about 25-100 mg, and preferably does not exceed 200 mg/d. A typical pediatric dose is about 0.1 mg/kg PO as initial dose, increasing, as tolerated, up to about 0.5-2 mg/d. Amitriptyline is preferably used for neuropathic pain in an adult dose of about 25-100 mg PO. See, e.g., Physicians' Desk Reference, 755, 1238, 1684 and 3495 (56^{th} ed., 2002).

Anticonvulsant drugs may also be used in embodiments of the invention. Examples of anticonvulsants include, but are not limited to, carbamazepine, oxcarbazepine (Trileptal®), gabapentin (Neurontin®), phenyloin, sodium valproate, clonazepam, topiramate, lamotrigine, zonisamide, and tiagabine. See, e.g., Physicians' Desk Reference, 2563 (57^{th} ed., 2003).

In one embodiment, a JNK Inhibitor and a second active agent are administered to a patient, preferably a mammal, more preferably a human, in a sequence and within a time interval such that the JNK Inhibitor can act together with the other agent to provide an increased benefit than if they were administered otherwise. For example, the second active agent can be administered at the same time or sequentially in any order at different points in time; however, if not administered at the same time, they should be administered sufficiently close in time so as to provide the desired therapeutic or prophylactic effect. In one embodiment, the JNK Inhibitor and the second active agent exert their effect at times which overlap. Each second active agent can be administered separately, in any appropriate form and by any suitable route. In other embodiments, the JNK Inhibitor is administered before, concurrently or after administration of the second active agent. Surgery can also be performed as a preventive measure or to relieve pain.

In various embodiments, the JNK Inhibitor and the second active agent are administered less than about 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, no more than 24 hours apart or no more than 48 hours apart. In other embodiments, the JNK Inhibitor and the second active agent are administered concurrently.

In other embodiments, the JNK Inhibitor and the second active agent are administered at about 2 to 4 days apart, at about 4 to 6 days apart, at about 1 week part, at about 1 to 2 weeks apart, or more than 2 weeks apart.

In certain embodiments, the JNK Inhibitor and optionally the second active agent are cyclically administered to a patient. Cycling therapy involves the administration of a first agent for a period of time, followed by the administration of a second agent and/or third agent for a period of time and repeating this sequential administration. Cycling therapy can reduce the development of resistance to one or more of the therapies, avoid or reduce the side effects of one of the therapies, and/or improve the efficacy of the treatment.

In certain embodiments, the JNK Inhibitor and optionally the second active agent are administered in a cycle of less than about 3 weeks, about once every two weeks, about once every 10 days or about once every week. One cycle can comprise the administration of a JNK Inhibitor and optionally the second active agent by infusion over about 90 minutes every cycle, about 1 hour every cycle, about 45 minutes every cycle. Each cycle can comprise at least 1 week of rest, at least 2 weeks of rest, at least 3 weeks of rest. The number of cycles administered is from about 1 to about 12 cycles, more typically from about 2 to about 10 cycles, and more typically from about 2 to about 8 cycles.

In yet other embodiments, the JNK Inhibitor is administered in metronomic dosing regimens, either by continuous infusion or frequent administration without extended rest periods. Such metronomic administration can involve dosing at constant intervals without rest periods. Typically the JNK Inhibitors, are used at lower doses. Such dosing regimens encompass the chronic daily administration of relatively low doses for extended periods of time. In preferred embodiments, the use of lower doses can minimize toxic side effects and eliminate rest periods. In certain embodiments, the JNK Inhibitor is delivered by chronic low-dose or continuous infusion ranging from about 24 hours to about 2 days, to about 1 week, to about 2 weeks, to about 3 weeks to about 1 month to about 2 months, to about 3 months, to about 4 months, to about 5 months, to about 6 months. The scheduling of such dose regimens can be optimized by the skilled artisan.

In other embodiments, courses of treatment are administered concurrently to a patient, i.e., individual doses of the second active agent are administered separately yet within a time interval such that the JNK Inhibitor can work together with the second active agent. For example, one component can be administered once per week in combination with the other components that can be administered once every two weeks or once every three weeks. In other words, the dosing regimens are carried out concurrently even if the therapeutics are not administered simultaneously or during the same day.

The second active agent can act additively or, more preferably, synergistically with the JNK Inhibitor. In one embodiment, a JNK Inhibitor is administered concurrently with one or more second active agents in the same pharmaceutical composition. In another embodiment, a JNK Inhibitor is administered concurrently with one or more second active agents in separate pharmaceutical compositions. In still another embodiment, a JNK Inhibitor is administered prior to or subsequent to administration of a second active agent. The invention contemplates administration of a JNK Inhibitor and a second active agent by the same or different routes of administration, e.g., oral and parenteral. In certain embodiments, when a JNK Inhibitor is administered concurrently with a second active agent that potentially produces adverse side effects including, but not limited to, toxicity, the second active agent can advantageously be administered at a dose that falls below the threshold that the adverse side effect is elicited.

4.2.2 Use With Physical Therapy or Psvchological Therapy

In still another embodiment, this invention encompasses a method of treating, preventing, modifying, and/or managing pain, which comprises administering a JNK Inhibitor in conjunction with physical therapy or psychological therapy.

Symptoms of pain include vasomotor dysfunction and movement disorders. A steady progression of gentle weight bearing to progressive active weight bearing is important in patients experiencing pain. Gradual desensitization to increasing sensory stimuli may also be helpful. Gradual increase in normalized sensation tends to reset the altered processing in the CNS. Physical therapy can thus play an important role in functional restoration. The goal of physical therapy is to gradually increase strength and flexibility.

It is believed that the combined use of a JNK Inhibitor and physical therapy may provide a unique treatment regimen that is unexpectedly effective in certain patients. Without being limited by theory, it is believed that a JNK Inhibitor may provide additive or synergistic effects when given concurrently with physical therapy.

Much pain literature notes a concomittent behavioral and psychiatric morbidities such as depression and anxiety. It is believed that the combined use of a JNK Inhibitor and psychological treatment may provide a unique treatment regimen that is unexpectedly effective in certain patients. Without being limited by theory, it is believed that a JNK Inhibitor may provide additive or synergistic effects when given concurrently with psychological therapy including, but not limited to, biofeedback, relaxation training, cognitive-behavioral therapy, and individual or family psychotherapy.

4.2.3 Use With Interventional Pain Management Techniques

In still another embodiment, this invention encompasses a method of treating, preventing, modifying, and/or managing pain, which comprises administering a JNK Inhibitor in conjunction with (e.g., before, during, or after) Pain Management interventional techniques. Examples of Pain Management interventional techniques include, but are not limited to, the use of sympathetic blocks, intravenous regional blocks, placement of dorsal column stimulators or placement of intrathecal infusion devices for analgesic medication delivery. Preferred Pain Management interventional techniques provides a selective neural blockade which interrupts the activity of the sympathetic nervous system in the region in which pain is experienced.

The combined use of the JNK Inhibitor and Pain Management interventional techniques may provide a unique treatment regimen that is unexpectedly effective in certain patients. Without being limited by theory, it is believed that a JNK Inhibitor may provide additive or synergistic effects when given concurrently with Pain Management interventional techniques. An example of Pain Management interventional techniques is intravenous regional block using BIER block with a variety of agents such as, but not limited to, local anesthetics such as bupivacaine, lidocaine, guanethidine, ketamine, bretylium, steroids, ketorolac, and reserpine. Perez, R. S., et al., J Pain Symptom Manage 21(6):511-26 (2001). For pain involving the upper extremities, a stellate (cervicothoracic) ganglion block may be used. The invention also encompasses the use of a somatic block, which involves continuous epidural infusion along with different variants of brachial plexus blocks. An axillary, supraclavicular, or infraclavicular approach of the somatic block may also be useful.

4.3 Pharmaceutical Compositions

The compositions comprising a JNK Inhibitor include bulk-drug compositions useful in the manufacture of pharmaceutical compositions (e.g., impure or non-sterile compositions) and pharmaceutical compositions (i.e., compositions that are suitable for administration to a patient) which can be used in the preparation of unit dosage forms. Such compositions optionally comprise an effective amount of a JNK Inhibitor or a combination of the JNK Inhibitors disclose herein and a pharmaceutically acceptable vehicle, excipient or carrier. Preferably, compositions of the invention comprise a prophylactically or therapeutically effective amount of JNK Inhibitor and optionally a second active agent, and a pharmaceutically acceptable carrier. In one embodiment, the second active agent is not an anti-cancer agent.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which a JNK Inhibitor is administered. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents can be used. When administered to a patient, the pharmaceutically acceptable vehicles are preferably sterile. Water can be the vehicle when the JNK Inhibitor is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propyleneglycol, water, ethanol and the like. The present compositions, if desired, can also contain minor amounts of wetting or emulsifying agents, or pH buffering agents.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the pharmaceutically acceptable vehicle is a capsule (see e.g., U.S. Pat. No. 5,698,155). Other examples of suitable pharmaceutical vehicles are described in “Remington's Pharmaceutical Sciences” by E. W. Martin.

In a preferred embodiment, the JNK Inhibitor and optionally the a therapeutic or prophylactic agent are formulated in accordance with routine procedures as pharmaceutical compositions adapted for intravenous administration to human beings. Typically, JNK Inhibitors for intravenous administration are solutions in sterile isotonic aqueous buffer. Where necessary, the compositions can also include a solubilizing agent. Compositions for intravenous administration can optionally include a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the JNK Inhibitor is to be administered by infusion, it can be dispensed, for example, with an infusion bottle containing sterile pharmaceutical grade water or saline. Where the JNK Inhibitor is administered by injection, an ampoule of sterile water for injection or saline can be provided so that the ingredients can be mixed prior to administration.

Compositions for oral delivery can be in the form of tablets, lozenges, aqueous or oily suspensions, granules, powders, emulsions, capsules, syrups, or elixirs, for example. Orally administered compositions can contain one or more optional agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving compound are also suitable for an orally administered JNK Inhibitor. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate can also be used. Oral compositions can include standard vehicles such as mannitol, lactose, starch, magnesium stearate, sodium saccharine, cellulose, magnesium carbonate, and the like. Such vehicles are preferably of pharmaceutical grade.

Further, the effect of the JNK Inhibitor can be delayed or prolonged by proper formulation. For example, a slowly soluble pellet of the JNK Inhibitor can be prepared and incorporated in a tablet or capsule. The technique can be improved by making pellets of several different dissolution rates and filling capsules with a mixture of the pellets. Tablets or capsules can be coated with a film which resists dissolution for a predictable period of time. Even the parenteral preparations can be made long-acting, by dissolving or suspending the compound in oily or emulsified vehicles which allow it to disperse only slowly in the serum.

4.4 Formulations

Pharmaceutical compositions for use in accordance with the present invention can be formulated in conventional manner using one or more physiologically acceptable carriers or excipients.

Thus, the JNK Inhibitor and optionally a second active agent, and their physiologically acceptable salts and solvates, can be formulated into pharmaceutical compositions for administration by inhalation or insufflation (either through the mouth or the nose) or oral, parenteral or mucosol (such as buccal, vaginal, rectal, sublingual) administration. In one embodiment, local or systemic parenteral administration is used.

For oral administration, the pharmaceutical compositions can take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well known in the art. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

Preparations for oral administration can be suitably formulated to give controlled release of the JNK Inhibitor.

For buccal administration the pharmaceutical compositions can take the form of tablets or lozenges formulated in conventional manner.

For administration by inhalation, the pharmaceutical compositions for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g., gelatin for use in an inhaler or insufflator can be formulated containing a powder mix of the compound and a suitable powder base such as lactose or starch.

The pharmaceutical compositions can be formulated for parenteral administration by injection, e.g., by bolus injection or continuous infusion. Formulations for injection can be presented in unit dosage form, e.g., in ampoules or in multi-dose containers, with an added preservative. The pharmaceutical compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

The pharmaceutical compositions can also be formulated in rectal compositions such as suppositories or retention enemas, e.g., containing conventional suppository bases such as cocoa butter or other glycerides.

In addition to the formulations described previously, the pharmaceutical compositions can also be formulated as a depot preparation. Such long acting formulations can be administered by implantation (for example subcutaneously or intramuscularly) or by intramuscular injection. Thus, for example, the pharmaceutical compositions can be formulated with suitable polymeric or hydrophobic materials (for example as an emulsion in an acceptable oil) or ion exchange resins, or as sparingly soluble derivatives, for example, as a sparingly soluble salt.

The invention also provides that a pharmaceutical composition can be packaged in a hermetically sealed container such as an ampoule or sachette indicating the quantity. In one embodiment, the pharmaceutical composition is supplied as a dry sterilized lyophilized powder or water free concentrate in a hermetically sealed container and can be reconstituted, e.g., with water or saline to the appropriate concentration for administration to a patient.

The pharmaceutical compositions can, if desired, be presented in a pack or dispenser device that can contain one or more unit dosage forms containing the active ingredient. The pack can for example comprise metal or plastic foil, such as a blister pack. The pack or dispenser device can be accompanied by instructions for administration.

In certain preferred embodiments, the pack or dispenser contains one or more unit dosage forms containing no more than the recommended dosage formulation as determined in the Physician 's Desk Reference (56^{th} ed. 2002, herein incorporated by reference in its entirety).

4.5 Routes of Administration

Methods of administering a JNK Inhibitor and optionally a second active agent include, but are not limited to, parenteral administration (e.g., intradermal, intramuscular, intraperitoneal, intravenous and subcutaneous), epidural, and mucosal (e.g., intranasal, rectal, vaginal, sublingual, buccal or oral routes). In a specific embodiment, the JNK Inhibitor and optionally the second active agent are administered intramuscularly, intravenously, or subcutaneously. The JNK Inhibitor and optionally the second active agent can also be administered by infusion or bolus injection and can be administered together with other biologically active agents. Administration can be local or systemic. The JNK Inhibitor and optionally the second active agent and their physiologically acceptable salts and solvates can also be administered by inhalation or insufflation (either through the mouth or the nose). In one embodiment, local or systemic parenteral administration is used.

In specific embodiments, it can be desirable to administer the JNK Inhibitor locally to the area in need of treatment. This can be achieved, for example, and not by way of limitation, by local infusion during surgery, topical application, e.g., in conjunction with a wound dressing after surgery, by injection, by means of a catheter, by means of a suppository, or by means of an implant, said implant being of a porous, nonporous, or gelatinous material, including membranes, such as sialastic membranes, or fibers. In one embodiment, administration can be by direct injection at the site (or former site) of an atherosclerotic plaque tissue.

Pulmonary administration can also be employed, e.g., by use of an inhaler or nebulizer, and formulation with an aerosolizing agent, or via perfusion in a fluorocarbon or synthetic pulmonary surfactant. In certain embodiments, the JNK Inhibitor can be formulated as a suppository, with traditional binders and vehicles such as triglycerides.

In another embodiment, the JNK Inhibitor can be delivered in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

In yet another embodiment, the JNK Inhibitor can be delivered in a controlled release system. In one embodiment, a pump can be used (see Langer, supra; Sefton, 1987, CRC Crit. Ref. Biomed. Eng. 14:201; Buchwald et al., 1980, Surgery 88:507 Saudek et al., 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105). In yet another embodiment, a controlled-release system can be placed in proximity of the target of the JNK Inhibitor, e.g., the liver, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) can be used.

4.6 Dosages

The amount of the JNK Inhibitor that is effective in the treatment, prevention, management and/or modification of pain can be determined by standard research techniques. For example, the dosage of the JNK Inhibitor which will be effective in the treatment, prevention, management and/or modification of pain can be determined by administering the JNK Inhibitor to an animal in a model such as, e.g., the animal models known to those skilled in the art. In addition, in vitro assays can optionally be employed to help identify optimal dosage ranges.

Selection of a particular effective dose can be determined (e.g., via clinical trials) by a skilled artisan based upon the consideration of several factors which will be known to one skilled in the art. Such factors include the disease to be treated or prevented, the symptoms involved, the patient's body mass, the patient's immune status and other factors known by the skilled artisan.

The precise dose to be employed in the formulation will also depend on the route of administration, and the seriousness of the pain, and should be decided according to the judgment of the practitioner and each patient's circumstances. Effective doses can be extrapolated from dose-response curves derived from in vitro or animal model test systems.

The dose of a JNK Inhibitor to be administered to a patient, such as a human, is rather widely variable and can be subject to independent judgment. It is often practical to administer the daily dose of a JNK Inhibitor at various hours of the day. However, in any given case, the amount of a JNK Inhibitor administered will depend on such factors as the solubility of the active component, the formulation used, patient condition (such as weight), and/or the route of administration.

In one embodiment, the general range of effective amounts of the JNK Inhibitor alone or in combination with a second active agent are from about 0.001 mg/day to about 1000 mg/day, more preferably from about 0.001 mg/day to 750 mg/day, more preferably from about 0.001 mg/day to 500 mg/day, more preferably from about 0.001 mg/day to 250 mg/day, more preferably from about 0.001 mg/day to 100 mg/day, more preferably from about 0.001 mg/day to 75 mg/day, more preferably from about 0.001 mg/day to 50 mg/day, more preferably from about 0.001 mg/day to 25 mg/day, more preferably from about 0.001 mg/day to 10 mg/day, more preferably from about 0.001 mg/day to 1 mg/day. In another embodiment, the general range of effective amounts of the JNK Inhibitor alone or in combination with a second active agent are from about 50 mg/day to about 1500 mg/day, more preferably from about 50 mg/day to 1000 mg/day, more preferably from about 100 mg/day to 400 mg/day. Of course, it is often practical to administer the daily dose of compound in portions, at various hours of the day. However, in any given case, the amount of compound administered will depend on such factors as the solubility of the active component, the formulation used, subject condition (such as weight), and/or the route of administration. In certain embodiments, the JNK Inhibitor can be administered daily, every other day, several times a week, weekly, bi-weekly or monthly.

4.7 Kits

The invention provides a pharmaceutical pack or kit comprising one or more containers containing a JNK Inhibitor and optionally one or more second active agents useful for the treatment, prevention, management and/or modification of pain. The invention also provides a pharmaceutical pack or kit comprising one or more containers containing one or more of the ingredients of the pharmaceutical compositions. Optionally associated with such container(s) can be a notice in the form prescribed by a governmental agency regulating the manufacture, use or sale of pharmaceuticals or biological products, which notice reflects approval by the agency of manufacture, use or sale for human administration; or instructions for the composition's use.

The present invention provides kits that can be used in the above methods. In one embodiment, a kit comprises a JNK Inhibitor, in one or more containers, and optionally one or more second active agents useful for the treatment, prevention or management of pain, in one or more additional containers.

### 5. EXAMPLES

The following examples illustrate certain aspects of the invention, but do not limit its scope.

The JNK Inhibitors can be tested for their ability to treat, prevent, manage and/or modify pain by any pain model well-known in the art. A variety of animal pain models are described in Hogan, Q., Regional Anesthesia and Pain Medicine 27(4):385-401 (2002), which is incorporated by reference herein in its entirety.

Examples of nociceptive pain models include the formalin test, the hot-plate test and the tail-flick test. These are useful models for injury-induced pain.

An illustrative example of the formalin test is set forth herein in Example 5.1. Briefly, formalin is injected into the plantar surface of a hind paw, and the effectiveness of the test compound is determined by recording the number of pain-associated behaviours observed over a period of time for a particular dose of the test compound. Abbott, F. et al. Pain 60:91-102 (1995).

An illustrative example of the hot-plate test is set forth herein in Example 5.2. Briefly, an animal is administered a test compound followed by observation of the length of time before the animal reacts to the heat stimulus of the hot plate. Malmberg, A. and Yaksh, T., Pain 60:83-90 (1995).

An illustrative example of the tail-flick test is set forth herein in Example 5.3. Briefly, an animal is administered a test compound followed by observation of the length of time before the animal reacts to the stimulus of a focused beam of light on its tail.

The most commonly used neuropathic pain models are the Bennett, Selzer, and Chung models. Siddall, P. J. and Munglani, R., Animal Models of Pain, pp 377-384 in Bountra, C., Munglani, R., Schmidt, W. K., eds. Pain: Current Understanding, Emerging Therapies and Novel Approaches to Drug Discovery, Marcel Dekker, Inc., New York, 2003. The Bennett and Selzer models are well-known and rapid to perform. The Chung model is robust for mechanical allodynia in most animals and is well characterized though complicated.

The capsaicin model as described herein in Example 5.4 may be appropriate for agents to be used to treat hyperalgesia and allodynia (e.g., vanilloid receptor 1 (VR1) antagonists and AMPA antagonists), whereas UV skin burn may be appropriate for bradykinin B1 receptor antagonists, cannabinoid agonists, and VR1 antagonists. Clinical applications of the capsaicin model have supported the antihyperalgesic effects of several clinically used drugs such as opioids, local anesthetics, ketamine and gabapentin. Visceral models have, as yet, unknown potential as hyperalgesic models and require validation.

These models represent a range of approaches to try and mimic some of the damage and dysfunction in clinical conditions. There are also animal models for diseases associated with pain, such as diabetic neuropathy or the new bone cancer and visceral pain models

A drawback with animal models is that they can only measure evoked pain. Hyperalgesia is most commonly measured. No animal model is able to measure spontaneous pain, which is of the most concnerning in connection with clinical pain states.

5.1 Formalin Test for the Measurement of Persistent Pain in Rats

Animals are injected with the a JNK Inhibitor or vehicle (controls) followed by the injection of formalin into the dorsal surface of the paw. The animal is observed to determine the number of times it flinches the injected paw, over a period of 60 minutes. This model allows for the evaluation of anti-nociceptive drugs in the treatment of pain.

Animals are contained in shoe box cages for the duration of the experiment. Formalin (50 μl; 0.5%) is injected into the dorsal surface of the rear, right paw, by placing the needle (28.5G) above the toes and below the ankle and inserting it beneath the surface of the skin. A timer is started immediately after the injection to mark the beginning of phase 1. The animal is observed for 10 minutes after injection and the number of times it flinches the injected paw are counted. Thirty minutes after the first formalin injection, phase 2 begins. Flinches are counted as in phase 1 for the next 20 minutes. A JNK Inhibitor is administered up to 24 hrs prior to the formalin test, by either oral, i.p., i.v. or s.c. routes of administration. Animals are repeated in the order they were treated. Immediately following the completion of the test periods, animals are euthanized by CO₂ asphyxiation in accordance with IACUC guidelines.

Any animal experiencing unanticipated events at any time point throughout this study is evaluated for veterinary intervention. Any animal that cannot recover with standard veterinary care is euthanized immediately by CO₂ asphyxiation in accordance with IACUC guidelines.

5.2 Hot-Plate Test for Measurement of Acute Pain in Rats

Animals are injected with a JNK Inhibitor or vehicle (controls) and then placed on the hot plate one at a time. Latency to respond to the heat stimulus is measured by the amount of time it takes for the animal to lick one of its paws. This model allows for the evaluation of anti-nociceptive drugs in the treatment of pain (See, Langerman et al., Pharmacol. Toxicol. Methods 34:23-27 (1995)).

Morphine treatment is used to determine the optimal hotplate temperature. Doses of 8 to 10 mg/kg morphine (i.p.) provide a near-maximal anti-nociceptive response in acute pain assays. The apparatus is set to the temperature at which this type of anti-nociceptive response is observed with these doses of morphine (approximately 55° C.). A JNK Inhibitor is dosed up to 24 hrs prior to the hot-plate test, by either oral, i.p., i.v. or s.c. routes of administration. When the post-treatment time has elapsed, individual testing of animals is begun. A single animal is placed on the hot plate and a stopwatch or timer is immediately started. The animal is observed until it shows a nociceptive response (e.g., licks its paw) or until the cut-off time of 30 seconds is reached (to minimize tissue damage that can occur with prolonged exposure to a heated surface). The animal is removed from the hot-plate and its latency time to respond is recorded. For animals that do not respond prior to the cut-off time, the cut-off time will be recorded as their response time. Animals are repeated in the order they were treated. Animals are euthanized immediately following the experiment by CO₂ asphyxiation in accordance with IACUC guidelines.

Any animal experiencing unanticipated events at any time point throughout this study is evaluated for veterinary intervention. Any animal that cannot recover with standard veterinary care is euthanized immediately by CO₂ asphyxiation in accordance with IACUC guidelines.

5.3 Tail-Flick Test for Measurement of Acute Pain in Rats

Animals are injected with the a JNK Inhibitor or vehicle (controls) and then a light beam is focused on the tail. Latency to respond to the stimulus is measured by the amount of time it takes for the animal to flick its tail. This model allows for the evaluation of anti-nociceptive drugs in the treatment of pain (See, Langerman et al., Pharmacol. Toxicol. Methods 34:23-27 (1995)).

A JNK Inhibitor is dosed up to 24 hrs prior to the tail flick test, by either oral, i.p., i.v. or s.c. routes of administration according with the IACUC guidelines. When the post-treatment time has elapsed, individual testing of animals is begun. A single animal is placed on a tail flick apparatus exposing the ventral tail surface to a focused light beam. Response latency is the time from the application of the light until the tail is flicked. The animal is observed until it shows a nociceptive response (e.g., tail flick) or until the cut-off time of 10 seconds is reached (to minimize tissue damage that can occur with prolonged exposure to a heated surface). The animal is removed from the light source, its latency time to respond is recorded and then the animal is euthanized immediately by CO₂ asphyxiation in accordance with IACUC guidelines. The light beam intensity is adjusted to produce a baseline latency of 2.5-4 seconds. For animals that do not respond prior to the cut-off time, the cut-off time is recorded as their response time. Animals are repeated in the order they were treated.

Any animal experiencing unanticipated events at any time point throughout this study is evaluated for veterinary intervention. Any animal that cannot recover with standard veterinary care is euthanized immediately by CO₂ asphyxiation in accordance with IACUC guidelines.

5.4 Model for Topical Capsaisin-Induced Thermal Allodynia

A model particularly useful for thermal allodynia is the topical capsaicin-induced thermal allodynia model. Butelman, E. R. et al., J. of Pharmacol. Exp. Therap. 306:1106-1114 (2003). This model is a modification of the warm water tail withdrawal model. Ko, M. C. et al., J. of Pharmacol. Exp. Therap. 289:378-385 (1999).

Briefly, monkeys sit in a custom made chair in a temperature-controlled room (20-22° C.). Their tails are shaved with standard clippers and tail withdrawal latencies are timed in 0.1 second increments up to a maximum of 20 seconds in both 38° C. and 42° C. water stimuli to provide a baseline. Following baseline determination, the tail is gently dried and degreased with an isopropyl alcohol pad.

Approximately 15 minutes before use, capsaicin is dissolved in a vehicle composed of 70% ethanol and 30% sterile water for a final capsaicin concentration of either 0.0013 or 0.004 M. The solution (0.3 mL) is slowly injected onto a gauze patch, saturating the patch and avoiding overflow. Within 30 seconds of the capsaicin solution being added to the patch, capsaicin patch is fastened to the tail with tape. After 15 minutes, the patch is removed and tail withdrawal testing in both 38° C. and 42° C. water stimuli is performed as described above.

Allodynia is detected as a decrease in tail withdrawal latency compared to the baseline measurements. To determine the ability of a JNK Inhibitor to decrease allodynia, a single dose of the compound is administered prior to (e.g., 15 minutes prior, 30 minutes prior, 60 minutes prior or 90 minutes prior) the application of the capsaicin patch. Alternatively, the allodynia reversal properties of a JNK Inhibitor can be determined by administering a single dose of the compound after application of the capsaicin patch (e.g., immediately after, 30 minutes after, 60 minutes after or 90 minutes after).

5.5 JNK Inhibitor Activity Assays

The ability of a JNK Inhibitor to inhibit JNK and accordingly, to be useful for the treatment, prevention, management and/or modification of pain, can be demonstrated using one or more of the following assays.

### 5.5.1 Example

### Biological Activity of 5-amino-anthra(9,1-cd)isothiazol-6-one

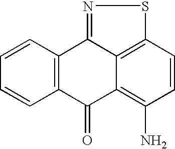

JNK Assay

To 10 μL of 5-amino-anthra(9,1-cd)isothiazol-6-one in 20% DMSO/80% dilution buffer containing of 20 mM HEPES (pH 7.6), 0.1 mM EDTA, 2.5 mM magnesium chloride, 0.004% Triton x100, 2 μg/mL leupeptin, 20 mM β-glycerolphosphate, 0.1 mM sodium vanadate, and 2 mM DTT in water was added 30 μL of 50-200 ng His6-JNK1, JNK2, or JNK3 in the same dilution buffer. The mixture was pre-incubated for 30 minutes at room temperature. Sixty microliter of 10 μg GST-c-Jun(1-79) in assay buffer consisting of 20 mM HEPES (pH 7.6), 50 mM sodium chloride, 0.1 mM EDTA, 24 mM magnesium chloride, 1 mM DTT, 25 mM PNPP, 0.05% Triton ×100, 11 μM ATP, and 0.5 μCi γ-32P ATP in water was added and the reaction was allowed to proceed for 1 hour at room temperature. The c-Jun phosphorylation was terminated by addition of 150 μL of 12.5% trichloroacetic acid. After 30 minutes, the precipitate was harvested onto a filter plate, diluted with 50 μL of the scintillation fluid and quantified by a counter. The IC₅₀ values were calculated as the concentration of 5-amino-anthra(9,1-cd)isothiazol-6-one at which the c-Jun phosphorylation was reduced to 50% of the control value. Compounds that inhibit JNK preferably have an IC₅₀ value ranging 0.01-10 μM in this assay. 5-Amino-anthra(9,1-cd)isothiazol-6-one has an IC₅₀ according to this assay of 1 μM for JNK2 and 400 nM for JNK3. The measured IC₅₀ value for 5-amino-anthra(9,1-cd)isothiazol-6-one, as measured by the above assay, however, shows some variability due to the limited solubility of 5-amino-anthra(9,1-cd)isothiazol-6-one in aqueous media. Despite the variability, however, the assay consistently does show that 5-amino-anthra(9,1-cd)isothiazol-6-one inhibits JNK. This assay demonstrates that 5-amino-anthra(9,1-cd)isothiazol-6-one, an illustrative JNK Inhibitor, inhibits JNK2 and JNK3 and, accordingly, is useful for the treatment, prevention, management and/or modification of pain.

Selectivity For JNK:

5-Amino-anthra(9,1-cd)isothiazol-6-one was also assayed for its inhibitory activity against several protein kinases, listed below, using techniques known to those skilled in art (See, e.g., Protein Phosphorylation, Sefton & Hunter, Eds., Academic Press, pp. 97-367, 1998). The following IC₅₀ values were obtained:

| Enzyme | IC₅₀ |
|---|---|
| p38-2 | >30,000 nM |
| MEK6 | >30,000 nM |
| LKK1 | >30,000 nM |
| IKK2 | >30,000 nM |

This assay shows that 5-amino-anthra(9,1-cd)isothiazol-6-one, an illustrative JNK Inhibitor, selectively inhibits JNK relative to other protein kinases and, accordingly, is a selective JNK Inhibitor. Therefore, 5-amino-anthra(9,1-cd)isothiazol-6-one, an illustrative JNK Inhibitor, is useful for the treatment, prevention, management and/or modification of pain.

Jurkat T-cell IL-2 Production Assay:

Jurkat T cells (clone E6-1) were purchased from the American Type Culture Collection of Manassas, Va. and maintained in growth media consisting of RPMI 1640 medium containing 2 mM L-glutamine (commercially available from Mediatech Inc. of Herndon, Va.), with 10% fetal bovine serum (commercially available from Hyclone Laboratories Inc. of Omaha, Nebr.) and penicillin/streptomycin. All cells were cultured at 37° C. in 95% air and 5% CO₂. Cells were plated at a density of 0.2×10⁶ cells per well in 200 μL of media. Compound stock (20 mM) was diluted in growth media and added to each well as a 10× concentrated solution in a volume of 25 μL, mixed, and allowed to pre-incubate with cells for 30 minutes. The compound vehicle (dimethylsulfoxide) was maintained at a final concentration of 0.5% in all samples. After 30 minutes the cells were activated with PMA (phorbol myristate acetate, final concentration 50 ng/mL) and PHA (phytohemagglutinin, final concentration 2 μg/mL). PMA and PHA were added as a 10× concentrated solution made up in growth media and added in a volume of 25 μL per well. Cell plates were cultured for 10 hours. Cells were pelleted by centrifugation and the media removed and stored at −20° C. Media aliquots are analyzed by sandwich ELISA for the presence of IL-2 as per the manufacturers instructions (Endogen Inc. of Woburn, Mass.). The IC₅₀ values were calculated as the concentration of 5-amino-anthra(9,1-cd)isothiazol-6-one at which the IL-2 production was reduced to 50% of the control value. Compounds that inhibit JNK preferably have an IC₅₀ value ranging from 0.1-30 μM in this assay. 5-Amino-anthra(9,1-cd)isothiazol-6-one has an IC₅₀ of 30 μM. The measured IC₅₀ value for 5-amino-anthra(9,1-cd)isothiazol-6-one, as measured by the above assay, however, shows some variability due to the limited solubility of 5-amino-anthra(9,1-cd)isothiazol-6-one in aqueous media. Despite the variability, however, the assay consistently does show that 5-amino-anthra(9,1-cd)isothiazol-6-one inhibits JNK.

This assay shows that 5-amino-anthra(9,1-cd)isothiazol-6-one, an illustrative JNK Inhibitor, inhibits IL-2 production in Jurkat T-cells and accordingly inhibits JNK. Therefore, 5-amino-anthra(9,1-cd)isothiazol-6-one, an illustrative JNK Inhibitor, is useful for the treatment, prevention, management and/or modification of pain.

[³H]Dopamine Cell Culture Assay:

Cultures of dopaminergic neurons were prepared according to a modification of the procedure described by Raymon and Leslie (J. Neurochem. 62:1015-1024, 1994). Time-mated pregnant rats were sacrificed on embyronic day 14-15 (crown rump length 11-12 mm) and the embryos removed by cesarean section. The ventral mesencephalon, containing the dopaminergic neurons, was dissected from each embryo. Tissue pieces from approximately 48 embryos were pooled and dissociated both enzymatically and mechanically. An aliquot from the resulting cell suspension was counted and the cells were plated in high glucose DMEM/F12 culture medium with 10% fetal bovine serum at a density of 1×10⁵ cells/well of a Biocoat poly-D-lysine-coated 96-well plate. The day following plating was considered 1 day in vitro (DUV). Cells were maintained in a stable environment at 37° C., 95% humidity, and 5% CO₂. A partial medium change was performed at 3 DIV. At 7 DIV, cells were treated with the neurotoxin, 6-hydroxydopamine (6-OHDA, 30 μM) in the presence and absence of 5-amino-anthra(9,1-cd)isothiazol-6-one. Cultures were processed for [³H]dopamine uptake 22 hours later.

[³H]Dopamine uptake is used as a measure of the health and integrity of dopaminergic neurons in culture (Prochiantz et al., PNAS 76: 5387-5391, 1979). It was used in these studies to monitor the viability of dopaminergic neurons following exposure to the neurotoxin 6-OHDA. 6-OHDA has been shown to damage dopaminergic neurons both in vitro and in vivo and is used to model the cell death observed in Parkinson's disease (Ungerstedt, U., Eur. J. Pharm., 5 (1968) 107-110 and Hefti et al., Brain Res., 195 (1980) 123-137). Briefly, cells treated with 6-OHDA in the presence and absence of 5-amino-anthra(9,1-cd)isothiazol-6-one were assessed in the uptake assay 22 hrs after exposure to 6-OHDA. Culture medium was removed and replaced with warm phosphate buffered saline (PBS) with calcium and magnesium, 10 μM pargyline, 1 mM ascorbic acid, and 50 nM [³H]dopamine. Cultures were incubated at 37° C. for 20 min. Radioactivity was removed and the cultures were washed 3× with ice cold PBS. To determine the intracellular accumulation of [³H]dopamine, cells were lysed with M-PER detergent and an aliquot was taken for liquid scintillation counting. The measured effect of 5-amino-anthra(9,1-cd) isothiazol-6-one on the intracellular accumulation of [³H]dopamine, as measured by the above assay, however, shows some variability due to the limited solubility of 5-amino-anthra(9,1-cd)isothiazol-6-one in aqueous media. Despite the variability, however, the assay consistently does show that 5-amino-anthra(9,1-cd)isothiazol-6-one protects rat ventral mesencephalan neurons from the toxic effects of 6-OHDA. Accordingly, 5-amino-anthra(9,1-cd)isothiazol-6-one, an illustrative JNK Inhibitor, is useful for the treatment, prevention, management and/or modification of pain.

Brain-Blood Plasma Distribution of 5-amino-anthra(9,1-cd)isothiazol-6-one In Vivo

5-Amino-anthra(9,1-cd)isothiazol-6-one was administered intravenously (10 mg/kg) into the veins of Sprague-Dawley rats. After 2 hr, blood samples were obtained from the animals and their vascular systems were perfused with approximately 100 mL of saline to rid their brains of blood. The brains were removed from the animals, weighed, and homogenized in a 50 mL conical tube containing 10 equivalents (w/v) of methanol/saline (1:1) using a Tissue Tearer (Fischer Scientific). The homogenized material was extracted by adding 600 μL of cold methanol to 250 μL of brain homogenate vortexed for 30 sec and subjected to centrifugation for 5 min. After centrifugation, 600 μL of the resulting supernatant was transferred to a clean tube and evaporated at room temperature under reduced pressure to provide a pellet. The resulting pellet was reconstituted in 250 μL of 30% aqueous methanol to provide a brain homogenate analysis sample. A plasma analysis sample was obtained using the brain homogenate analysis sample procedure described above by substituting plasma for brain homogenate. Standard plasma samples and standard brain homogenate samples containing known amounts of 5-amino-anthra(9,1-cd)isothiazol-6-one were also prepared by adding 5 μL of serial dilutions (50:1) of a solution of 5-amino-anthra(9,1-cd)isothiazol-6-one freshly prepared in cold ethanol to 250 μ/L of control rat plasma (Bioreclamation of Hicksville, N.Y.) or control brain homogenate. The standard plasma samples and standard brain homogenate samples were then subjected to the same extraction by protein precipitation, centrifugation, evaporation, and reconstitution procedure used for the brain homogenate to provide brain homogenate standard analysis samples and plasma standard analysis samples. The brain homogenate analysis samples, plasma analysis samples, and standard analysis samples were analyzed and compared using HPLC by injecting 100 μL of a sample onto a 5 μm C-18 Luna column (4.6 mm×150 mm, commercially available from Phenomenex of Torrance, Calif.) and eluting at 1 mL/min with a linear gradient of 30% aqueous acetonitrile containing 0.1% trifluoroacetic acid to 90% aqueous acetonitrile containing 0.1% trifluoroacetic acid over 8 minutes and holding at 90% aqueous acetonitrile containing 0.1% trifluoroacetic acid for 3 min. with absorbance detection at 450 nm. Recovery of 5-amino-anthra(9,1-cd)isothiazol-6-one was 56±5.7% for plasma and 42+6.2% for the brain. The concentration of 5-amino-anthra(9,1-cd) isothiazol-6-one in the brain and plasma was determined by comparing HPLC chromatograms obtained from the brain homogenate analysis samples and plasma analysis samples to standard curves constructed from analysis of the brain homogenate standard analysis samples and the plasma standard analysis samples, respectively. Results from this study show that 5-amino-anthra(9,1-cd)isothiazol-6-one, following intravenous administration, crosses the blood-brain barrier to a significant extent. In particular, brain-drug concentrations were approximately 65 nmole/g and plasma concentrations were approximately 7 μM at 2 hr post-dose, resulting in a brain-plasma concentration ratio of approximately 9-fold (assuming 1 g of brain tissue is equivalent to 1 mL of plasma). This example shows that 5-amino-anthra(9,1-cd)isothiazol-6-one, an illustrative JNK Inhibitor, has enhanced ability to cross the blood-brain barrier. In addition, this example shows that the JNK Inhibitors, in particular 5-amino-anthra(9,1-cd)isothiazol-6-one, can cross the blood-brain barrier when administered to a patient.

It will be appreciated that, although specific embodiments of the invention have been described herein for purposes of illustration, the invention described and claimed herein is not to be limited in scope by the specific embodiments herein disclosed. These embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

A number of references have been cited, the entire disclosure of which are incorporated herein by reference in their entirety.

What is claimed is:

1. A method for treating, preventing, managing and/or modifying pain in a patient, comprising administering to a patient in need thereof an effective amount of a JNK Inhibitor or a pharmaceutically acceptable salt, solvate or stereoisomer thereof.

2. A method for treating, preventing, managing and/or modifying pain in a patient, comprising administering to a patient in need thereof an effective amount of a compound having the following formula: 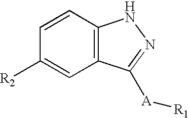
or a pharmaceutically acceptable salt, solvate or stereoisomer thereof, wherein:
A is a direct bond, —(CH₂)ₐ—, —(CH₂)_{b}CH═CH(CH₂)_{c}—, or —(CH₂)_{b}C≡C(CH₂)_{c}—;
R₁ is aryl, heteroaryl or heterocycle fused to phenyl, each being optionally substituted with one to four substituents independently from R₃;
R₂ is —R₃, —R₄, —(CH₂)_{b}C(═O)R₅, —(CH₂)_{b}C(═O)OR₅, —(CH₂)_{b}C(═O)NR₅R₆, —(CH₂)_{b}C(═O)NR₅(CH₂)_{c}C(═O)R₆, —(CH₂)_{b}NR₅C(═O)R₆, —(CH₂)_{b}NR₅C(═O)NR₆R₇, —(CH₂)_{b}NR₅R₆, —(CH₂)_{b}OR₅, —(CH₂)_{b}SO_{d}R₅ or —(CH₂)_{b}SO₂NR₅R₆;
a is 1, 2, 3, 4, 5 or 6;
b and c are the same or different and at each occurrence independently 0, 1, 2, 3 or 4;
d is at each occurrence 0, 1 or 2;
R₃ is at each occurrence independently halogen, hydroxy, carboxy, alkyl, alkoxy, haloalkyl, acyloxy, thioalkyl, sulfinylalkyl, sulfonylalkyl, hydroxyalkyl, aryl, substituted aryl, arylalkyl, heterocycle, heterocycloalkyl, —C(═O)OR₈, —OC(═O)R₈, —C(═O)NR₈R₉, —C(═O)NR₈OR₉, —SO₂NR₈R₉, —NR₈SO₂R₉, —CN, —NO₂, —NR₈R₉, —NR₈C(═O)R₉, —NR₈C(═O)(CH₂)_{b}OR₉, —NR₈C(═O)(CH₂)_{b}R₉, —O(CH₂)_{b}NR₈R₉, or heterocycle fused to phenyl;
R₄ is alkyl, aryl, arylalkyl, heterocycle or heterocycloalkyl, each being optionally substituted with one to four substituents independently from R₃, or R₄ is halogen or hydroxy;
R₅, R₆ and R₇ are the same or different and at each occurrence independently hydrogen, alkyl, aryl, arylalkyl, heterocycle or heterocycloalkyl, wherein each of R₅, R₆ and R₇ are optionally substituted with one to four substituents independently selected from R₃; and
R₈ and R₉ are the same or different and at each occurrence independently hydrogen, alkyl, aryl, arylalkyl, heterocycle, or heterocycloalkyl, or R₈ and R₉ taken together with the atom or atoms to which they are bonded form a heterocycle, wherein each of R₈, R₉, and R₈ and R₉ taken together to form a heterocycle are optionally substituted with one to four substituents independently selected from R₃.

3. A method for treating, preventing, managing and/or modifying pain in a patient, comprising administering to a patient in need thereof an effective amount of a compound having the following formula: 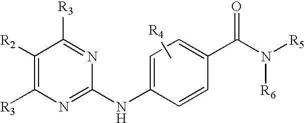
or a pharmaceutically acceptable salt, solvate or stereoisomer thereof, wherein:
R₁ is aryl or heteroaryl optionally substituted with one to four substituents independently selected from R₇;
R₂ is hydrogen;
R₃ is hydrogen or lower alkyl;
R₄ represents one to four optional substituents, wherein each substituent is the same or different and independently halogen, hydroxy, lower alkyl or lower alkoxy;
R₅ and R₆ are the same or different and independently —R₈, —(CH₂)ₐC(═O)R₉, —(CH₂)ₐC(═O)OR₉, —(CH₂)ₐC(═O)NR₉R₁₀, —(CH₂)ₐC(═O)NR₉(CH₂)_{b}C(═O)R₁₀, (CH₂)ₐNR₉C(═O)R₁₀, (CH₂)ₐNR₁₁C(═O)NR₉R₁₀, —(CH₂)ₐNR₉R₁₀, —(CH₂)ₐOR₉, —(CH₂)ₐSO_{c}R₉ or —(CH₂)ₐSO₂NR₉R₁₀;
or R₅ and R₆ taken together with the nitrogen atom to which they are attached to form a heterocycle or substituted heterocycle;
R₇ is at each occurrence independently halogen, hydroxy, cyano, nitro, carboxy, alkyl, alkoxy, haloalkyl, acyloxy, thioalkyl, sulfinylalkyl, sulfonylalkyl, hydroxyalkyl, aryl, arylalkyl, heterocycle, heterocycloalkyl, —C(═O)OR₈, —OC(═O)R₈, —C(═O)NR₈R₉, —C(═O)NR₈OR₉, —SO_{c}R₈, —SO_{c}NR₈R₉, —NR₈SO_{c}R₉, —NR₈R₉, —NR₈C(═O)R₉, —NR₉C(═O)(CH₂)_{b}OR₉, —NR₈C(═O)(CH₂)_{b}R₉, —O(CH₂)_{b}NRSR₉, or heterocycle fused to phenyl;
R₈, R₉, R₁₀ and R₁₁ are the same or different and at each occurrence independently hydrogen, alkyl, substituted alkyl, aryl, arylalkyl, heterocycle or heterocycloalkyl.;
or R₈ and R₉ taken together with the atom or atoms to which they are attached to form a heterocycle;
a and b are the same or different and at each occurrence independently 0, 1, 2, 3 or 4; and
c is at each occurrence 0, 1 or 2.

4. A method for treating, preventing, managing and/or modifying pain in a patient, comprising administering to a patient in need thereof an effective amount of a compound having the following formula: 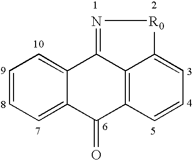
or a pharmaceutically acceptable salt, solvate or stereoisomer thereof,
wherein R₀ is —O—, —S—, —S(O)—, —S(O)₂—, NH or —CH₂—;
the compound being (i) unsubstituted, (ii) monosubstituted and having a first substituent, or (iii) disubstituted and having a first substituent and a second substituent;
the first or second substituent, when present, is at the 3, 4, 5, 7, 8, 9, or 10 position, wherein the first and second substituent, when present, are independently alkyl, hydroxy, halogen, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono-alkylaminoalkoxy, di-alkylaminoalkoxy, or a group represented by formula (a), (b), (c), (d), (e), or (f): 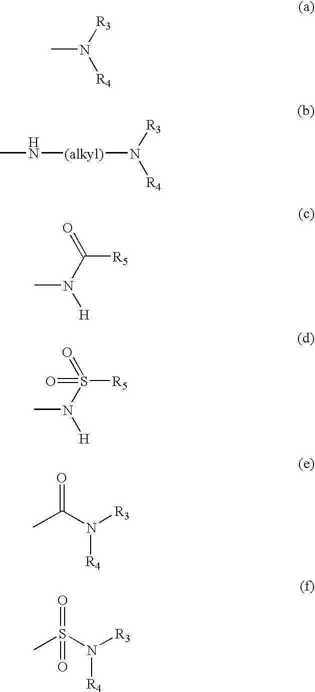
wherein R₃ and R₄ are taken together and represent alkylidene or a heteroatom-containing cyclic alkylidene or R₃ and R₄ are independently hydrogen, alkyl, cycloalkyl, aryl, arylalkyl, cycloalkylalkyl, aryloxyalkyl, alkoxyalkyl, aminoalkyl, mono-alkylaminoalkyl, or di-alkylaminoalkyl; and
R₅ is hydrogen, alkyl, cycloalkyl, aryl, arylalkyl, cycloalkylalkyl, alkoxy, alkoxyalkyl, alkoxycarbonylalkyl, amino, mono-alkylamino, di-alkylamino, arylamino, arylalkylamino, cycloalkylamino, cycloalkylalkylamino, aminoalkyl, mono-alkylaminoalkyl, or di-alkylaminoalkyl.

5. The method of claim 2 wherein A is a direct bond.

6. The method of claim 2 wherein A is —(CH₂)ₐ—.

7. The method of claim 2 wherein A is —(CH₂)_{b}CH═CH(CH₂)_{c}—.

8. The method of claim 2 wherein A is —(CH₂)_{b}C≡C(CH₂)_{c}—.

9. The method of claim 2 wherein the compound has the following formula: 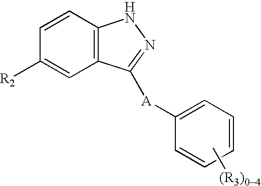
or a pharmaceutically acceptable salt, solvate or stereoisomer thereof, wherein:
A is a direct bond, —(CH₂)ₐ—, —(CH₂)_{b}CH═CH(CH₂)_{c}—, or —(CH₂)_{b}C≡C(CH₂)_{c}—;
R₁ is aryl, heteroaryl or heterocycle fused to phenyl, each being optionally substituted with one to four substituents independently from R₃;
R₂ is —R₃, —R₄, —(CH₂)_{b}C(═O)R₅, —(CH₂)_{b}C(═O)OR₅, —(CH₂)_{b}C(═O)NR₅R₆, —(CH₂)_{b}C(═O)NR₅(CH₂)_{c}C(═O)R₆, —(CH₂)_{b}NR₅C(═O)R₆, —(CH₂)_{b}NR₅C(═O)NR₆R₇, —(CH₂)_{b}NR₅R₆, —(CH₂)_{b}OR₅, —(CH₂)_{b}SO_{d}R₅ or —(CH₂)_{b}SO₂NR₅R₆;
a is 1, 2, 3, 4, 5 or 6;
b and c are the same or different and at each occurrence independently selected from 0, 1, 2, 3 or 4;
d is at each occurrence 0, 1 or 2;
R₃ is at each occurrence independently halogen, hydroxy, carboxy, alkyl, alkoxy, haloalkyl, acyloxy, thioalkyl, sulfinylalkyl, sulfonylalkyl, hydroxyalkyl, aryl, arylalkyl, heterocycle, heterocycloalkyl, —C(═O)OR₈, —OC(═O)R₈, —C(═O)NR₈R₉, —C(═O)NR₈OR₉, —SO₂NR₈R₉, —NR₈SO₂R₉, —CN, —NO₂, —NR₈R₉, —NR₈C(═O)R₉, —NR₈C(═O)(CH₂)_{b}OR₉, —NR₈C(═O)(CH₂)_{b}R₉, —O(CH₂)_{b}NR₉R₉, or heterocycle fused to phenyl;
R₄ is alkyl, aryl, arylalkyl, heterocycle or heterocycloalkyl, each being optionally substituted with one to four substituents independently from R₃, or R₄ is halogen or hydroxy;
R₅, R₆ and R₇ are the same or different and at each occurrence independently hydrogen, alkyl, aryl, arylalkyl, heterocycle or heterocycloalkyl, wherein each of R₅, R₆ and R₇ are optionally substituted with one to four substituents independently selected from R₃; and
R₈ and R₉ are the same or different and at each occurrence independently hydrogen, alkyl, aryl, arylalkyl, heterocycle, or heterocycloalkyl, or R₈ and R₉ taken together with the atom or atoms to which they are bonded form a heterocycle, wherein each of R₈, R₉, and R₈ and R₉ taken together to form a heterocycle are optionally substituted with one to four substituents independently selected from R₃.

10. The method of claim 2 wherein the compound has the following formula: 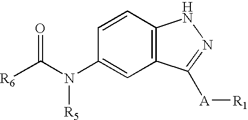
or a pharmaceutically acceptable salt, solvate or stereoisomer thereof, wherein:
A is a direct bond, —(CH₂)ₐ—, —(CH₂)_{b}CH═CH(CH₂)_{c}—, or —(CH₂)_{b}C═C(CH₂)_{c}—;
R₁ is aryl, heteroaryl or heterocycle fused to phenyl, each being optionally substituted with one to four substituents independently from R₃;
R₂ is —R₃, —R₄, —(CH₂)_{b}C(═O)R₅, —(CH₂)_{b}C(═O)OR₅, —(CH₂)_{b}C(═O)NR₅R₆, —(CH₂)_{b}C(═O)NR₅(CH₂)_{c}C(═O)R₆, —(CH₂)_{b}NR₅C(═O)R₆, —(CH₂)_{b}NR₅C(═O)NR₆R₇, —(CH₂)_{b}NR₅R₆, —(CH₂)_{b}OR₅, —(CH₂)_{b}SO_{d}R₅ or —(CH₂)_{b}SO₂NR₅R₆;
a is 1, 2, 3, 4, 5 or 6;
b and c are the same or different and at each occurrence independently 0, 1, 2, 3 or 4;
d is at each occurrence 0, 1 or 2;
R₃ is at each occurrence independently halogen, hydroxy, carboxy, alkyl, alkoxy, haloalkyl, acyloxy, thioalkyl, sulfinylalkyl, sulfonylalkyl, hydroxyalkyl, aryl, arylalkyl, heterocycle, heterocycloalkyl, —C(═O)OR₈, —OC(═O)R₈, —C(═O)NR₈R₉, —C(═O)NR₈OR₉, —SO₂NR₈R₉, —NR₈SO₂R₉, —CN, —NO₂, —NR₈R₉, —NR₈C(═O)R₉, —NR₈C(═O)(CH₂)_{b}OR₉, —NR₈C(═O)(CH₂)_{b}R₉, —O(CH₂)_{b}NR₈R₉, or heterocycle fused to phenyl;
R₄ is alkyl, aryl, arylalkyl, heterocycle or heterocycloalkyl, each being optionally substituted with one to four substituents independently from R₃, or R₄ is halogen or hydroxy;
R₅, R₆ and R₇ are the same or different and at each occurrence independently hydrogen, alkyl, aryl, arylalkyl, heterocycle or heterocycloalkyl, wherein each of R₅, R₆ and R₇ are optionally substituted with one to four substituents independently selected from R₃; and
R₈ and R₉ are the same or different and at each occurrence independently hydrogen, alkyl, aryl, arylalkyl, heterocycle, or heterocycloalkyl, or R₈ and R₉ taken together with the atom or atoms to which they are bonded form a heterocycle, wherein each of R₈, R₉, and R₈ and R₉ taken together to form a heterocycle are optionally substituted with one to four substituents independently selected from R₃.

11. The method of claim 2 wherein the compound has the following formula: 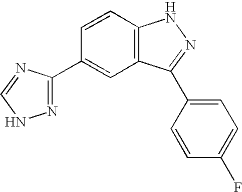
or a pharmaceutically acceptable salt, solvate or stereoisomer thereof.

12. The method of claim 3, wherein the compound has the following formula: 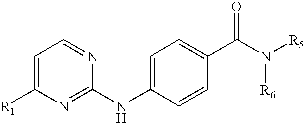
or a pharmaceutically acceptable salt, solvate or stereoisomer thereof, wherein:
R₁ is aryl or heteroaryl optionally substituted with one to four substituents independently selected R₇;
R₂ is hydrogen;
R₃ is hydrogen or lower alkyl;
R₄ represents one to four optional substituents, wherein each substituent is the same or different and independently halogen, hydroxy, lower alkyl or lower alkoxy;
R₅ and R₆ are the same or different and independently —R₈, —(CH₂)ₐC(═O)R₉, —(CH₂)ₐC(═O)OR₉, —(CH₂)ₐC(═O)NR₉R₁₀, —(CH₂)ₐC(═O)NR₉(CH₂)_{b}C(═O)R₁₀, —(CH₂)ₐNR₉C(═O)R₁₀, (CH₂)ₐNR₁₁C(═O)NR₉R₁₀, —(CH₂)ₐNR₉R₁₀, —(CH₂)ₐOR₉, —(CH₂)ₐSO_{c}R₉ or —(CH₂)ₐSO₂NR₉R₁₀;
or R₅ and R₆ taken together with the nitrogen atom to which they are attached to form a heterocycle or substituted heterocycle;
R₇ is at each occurrence independently halogen, hydroxy, cyano, nitro, carboxy, alkyl, alkoxy, haloalkyl, acyloxy, thioalkyl, sulfinylalkyl, sulfonylalkyl, hydroxyalkyl, aryl, arylalkyl, heterocycle, heterocycloalkyl, —C(═O)OR₈, —OC(═O)R₈, —C(═O)NR₈R₉, —C(═O)NR₈OR₉, —SO_{c}R₈, —SO_{c}NR₈R₉, —NR₈SO_{c}R₉, —NR₈R₉, —NR₈C(═O)R₉, —NR₈C(═O)(CH₂)_{b}OR₉, —NR₈C(═O)(CH₂)_{b}R₉, —O(CH₂)_{b}NR₈R₉, or heterocycle fused to phenyl; R₈, R₉, R₁₀ and R₁₁ are the same or different and at each occurrence independently hydrogen, alkyl, substituted alkyl, aryl, substituted aryl, arylalkyl, heterocycle, heterocycloalkyl;
or R₈ and R₉ taken together with the atom or atoms to which they are attached to form a heterocycle;
a and b are the same or different and at each occurrence independently 0, 1, 2, 3 or 4; and
c is at each occurrence 0, 1 or 2.

13. The method of claim 3, wherein the compound has the following formula: 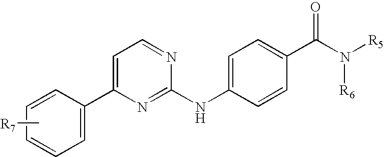
or a pharmaceutically acceptable salt, solvate or stereoisomer thereof, wherein:
R₁ is aryl or heteroaryl optionally substituted with one to four substituents independently from R₇;
R₂ is hydrogen;
R₃ is hydrogen or lower alkyl;
R₄ represents one to four optional substituents, wherein each substituent is the same or different and independently halogen, hydroxy, lower alkyl or lower alkoxy;
R₅ and R₆ are the same or different and independently —R₈, —(CH₂)ₐC(═O)R₉, —(CH₂)ₐC(═O)OR₉, —(CH₂)ₐC(═O)NR₉R₁₀, —(CH₂)ₐC(═O)NR₉(CH₂)_{b}C(═O)R₁₀, —(CH₂)ₐNR₉C(═O)R₁₀, (CH₂)ₐNR₁₁C(═O)NR₉R₁₀, —(CH₂)ₐNR₉R₁₀, —(CH₂)OR₉, —(CH₂)ₐSO_{c}R₉ or —(CH₂)ₐSO₂NR₉R₁₀;
or R₅ and R₆ taken together with the nitrogen atom to which they are attached to form a heterocycle or substituted heterocycle;
R₇ is at each occurrence independently halogen, hydroxy, cyano, nitro, carboxy, alkyl, alkoxy, haloalkyl, acyloxy, thioalkyl, sulfinylalkyl, sulfonylalkyl, hydroxyalkyl, aryl, arylalkyl, heterocycle, heterocycloalkyl, —C(═O)OR₈, —OC(═O)R₈, —C(═O)NR₈R₉, —C(═O)NR₈OR₉, —SO_{c}R₈, —SO_{c}NR₈R₉, —NR₈SO_{c}R₉, —NR₈R₉, —NR₈C(═O)R₉, —NR₈C(═O)(CH₂)_{b}OR₉, —NR₈C(═O)(CH₂)_{b}R₉, —O(CH₂)_{b}NR₈R₉, or heterocycle fused to phenyl;
R₈, R₉, R₁₀ and R₁₁ are the same or different and at each occurrence independently hydrogen, alkyl, aryl, arylalkyl, heterocycle, heterocycloalkyl;
or R₈ and R₉ taken together with the atom or atoms to which they are attached to form a heterocycle;
a and b are the same or different and at each occurrence independently 0, 1, 2, 3 or 4; and
c is at each occurrence 0, 1 or 2.

14. The method of claim 3, wherein the compound has the following formula: 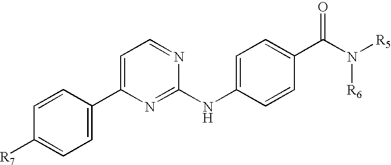
or a pharmaceutically acceptable salt, solvate or stereoisomer thereof, wherein:
R₁ is aryl or heteroaryl optionally substituted with one to four substituents independently from R₇;
R₂ is hydrogen;
R₃ is hydrogen or lower alkyl;
R₄ represents one to four optional substituents, wherein each substituent is the same or different and independently from halogen, hydroxy, lower alkyl or lower alkoxy;
R₅ and R₆ are the same or different and independently —R₈, —(CH₂)ₐC(═O)R₉, —(CH₂)ₐC(═O)OR₉, —(CH₂)ₐC(═O)NR₉R₁₀, —(CH₂)ₐC(═O)NR₉(CH₂)_{b}C(═O)R₁₀, —(CH₂)ₐNR₉C(═O)R₁₀, (CH₂)ₐNR₁₁C(═O)NR₉R₁₀, (CH₂)ₐNR₉R₁₀, —(CH₂)ₐOR₉, —(CH₂)ₐSO_{c}R₉ or —(CH₂)ₐSO₂NR₉R₁₀;
or R₅ and R₆ taken together with the nitrogen atom to which they are attached to form a heterocycle;
R₇ is at each occurrence independently halogen, hydroxy, cyano, nitro, carboxy, alkyl, alkoxy, haloalkyl, acyloxy, thioalkyl, sulfinylalkyl, sulfonylalkyl, hydroxyalkyl, aryl, arylalkyl, heterocycle, heterocycloalkyl, —C(═O)OR₈, —OC(═O)R₈, —C(═O)NR₈R₉, —C(═O)NR₈OR₉, —SO_{c}R₈, —SO_{c}NR₈R₉, —NR₈SO_{c}R₉, —NR₈R₉, —NR₈C(═O)R₉, —NR₈C(═O)(CH₂)_{b}OR₉, —NR₈C(═O)(CH₂)_{b}R₉, —O(CH₂)_{b}NR₈R₉, or heterocycle fused to phenyl;
R₈, R₉, R₁₀ and R₁₁ are the same or different and at each occurrence independently hydrogen, alkyl, substituted alkyl, aryl, arylalkyl, heterocycle, heterocycloalkyl;
or R₈ and R₉ taken together with the atom or atoms to which they are attached to form a heterocycle;
a and b are the same or different and at each occurrence independently 0, 1, 2, 3 or 4; and
c is at each occurrence 0, 1 or 2.

15. The method of claim 4, wherein R₀ is —O—.

16. The method of claim 4, wherein R₀ is —S—.

17. The method of claim 4, wherein R₀ is —S(O)—.

18. The method of claim 4, wherein R₀ is —S(O)₂—.

19. The method of claim 4, wherein R₀ is NH.

20. The method of claim 4, wherein R₀ is CH₂—.

21. The method of claim 4, wherein the compound has the following formula: 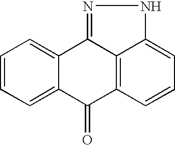
or a pharmaceutically acceptable salt, solvate or stereoisomer thereof.

22. The method of claim 1, further comprising administering a second active agent.

23. The method of claim 2, further comprising administering a second active agent.

24. The method of claim 3, further comprising administering a second active agent.

25. The method of claim 4, further comprising administering a second active agent.

26. The method of claim 22, wherein the second active agent is an antidepressant, antihypertensive, anxiolytic, calcium channel blocker, muscle relaxant, non-narcotic analgesic, anti-inflammatory agent, cox-2 inhibitor, alpha-adrenergic receptor agonist or antagonist, ketamine, anesthetics, immunomodulatory agent, immunosuppressive agent, corticosteroid, hyperbaric oxygen, anticonvulsant, an IMiD®, a SelCID®, or a combination thereof.

27. The method of claim 22, wherein the second active agent is gabapentin, thalidomide, salicyclic acid acetate, ketamine, celocoxib, carbamazepine, oxcarbazepine, phenyloin, sodium valproate, prednisone, nifedipine, clonidine, oxycodone, meperidine, morphine sulfate, hydromorphone, fentanyl, acetaminophen, ibuprofen, naproxen sodium, griseofulvin, amitriptyline, imipramine, doxepin, or a pharmaceutically acceptable salt, solvate or stereoisomer thereof.

28. The method of claim 1, wherein the pain is complex regional pain syndrome.

29. The method of claim 28, wherein the complex regional pain syndrome is type I or type II.

30. The method of claim 28, wherien the complex regional pain syndrome is stage I, stage II or stage III of complex regional pain syndrome type I.

31. The method of claim 28, wherien the complex regional pain syndrome is pain, autonomic dysfunction, trigeminal neuralgia, post-herpetic neuralgia, cancer-related pain, phantom limb pain, fibromyalgia, chronic fatigue syndrome, radiculopathy, inability to initiate movement, weakness, tremor, muscle spasm, dytonia, dystrophy, atrophy, edema, stiffness, joint tenderness, increased sweating, sensitivity to temperature, light touch (allodynia), color change to the skin, hyperthermic or hypothermic, increased nail and hair growth, early bony changes, hyperhidrotic with livedo reticularis or cyanosis, lost hair, ridged, cracked or brittle nails, dry hand, diffuse osteoporosis, irreversible tissue damage, thin and shiny skin, joint contractures, marked bone demineralization, diabetic neuropathy, luetic neuropathy, painful neuropathy induced iatrogenically by a drug, or another painful neuropathic condition.

32. The method of claim 1, wherein the pain is nociceptive pain.

33. The method of claim 32, wherein the nociceptive pain is associated with a cut or contusion of the skin; a chemical or thermal burn; osteoarthritis; rheumatoid arthritis; or tendonitis.

34. The method of claim 1, wherein the pain is neuropathic pain.

35. The method of claim 34, wherein the neuropathic pain is associated with stroke, diabetic neuropathy, luetic neuropathy, postherpetic neuralgia, trigeminal neuralgia, fibromyalgia, or painful neuropathy induced iatrogenically by a drug.

36. A pharmaceutical composition comprising an effective amount of a JNK Inhibitor and an antidepressant, antihypertensive agent, anxiolytic agent, calcium channel blocker, muscle relaxant, non-narcotic analgesic, anti-inflammatory agent, cox-2 inhibitor, alpha-adrenergic receptor agonist or antagonist, ketamine, an anesthetic, an immunomodulatory agent, an immunosuppressive agent, a corticosteroid, hyperbaric oxygen, an anticonvulsant, an IMiD®, a SelCID®, or a combination thereof.
